(19) United States
(12) Patent Application Publication
(10) Pub. No.: US 2024/0350035 A1
(43) Pub. Date: Oct. 24, 2024

(54) SYSTEM AND METHOD FOR QUANTITATIVE GAIT ASSESSMENT

(71) Applicant: JASPER MEDTECH PTY LTD, Sydney (AU)

(72) Inventors: Ralph MOBBS, Sydney (AU); Dineth FONSEKA, Sydney (AU); Monish MAHARAJ, Sydney (AU); Pragadesh NATARAJAN, Sydney (AU); Luke SY, Sydney (AU)

(21) Appl. No.: 18/686,171

(22) PCT Filed: Aug. 23, 2022
(86) PCT No.: PCT/AU2022/050953

§ 371 Date: Feb. 23, 2024

(30) Foreign Application Priority Data
Aug. 23, 2021 (AU) 2021106529
Aug. 25, 2021 (AU) 2021107366

(51) Int. Cl. A61B 5/11; A61B 5/00

(52) U.S. Cl. CPC A61B 5/112; A61B 5/1117; A61B 5/7275; A61B 2562/0219; A61B 2562/0223

(57) ABSTRACT

There is provided a system for determining a quantitative gait score, the system comprising an accelerometer, a magnetometer, a gyroscope; and a processor configured to receive output signals from the accelerometer, a magnetometer, a gyroscope and analyse the signals to determine the quantitative gait score for the subject from one or any combination of daily step count; step cadence; step time; step time asymmetry; step length; stride length; step length asymmetry; single support time variability; walking orientation randomness metric (WORM Score); and gait velocity or gait velocity variation. Methods of determining the quantitative gait score are also provided.

### TECHNICAL FIELD

The present invention relates to a system and method for determining quantitative gait scores from combinations of gait metrics. In particular the invention relates to quantitative gait scores termed the ‘Simplified Mobility Score’, ‘Immediate Mobility Score’, the ‘Gait Symmetry Index’, and the ‘Combined Mobility Score’.

### CROSS REFERENCE TO RELATED APPLICATIONS

This application claims Australian Innovation Patent No 2021106529 titled ‘System and method for quantitative gait assessment’, filed 23 Aug. 2021, and Australian Innovation Patent No 2021107366 titled ‘System and method for determining a combined mobility score’, filed 25 Aug. 2021. The disclosure of both applications is incorporated by reference in their entireties.

### BACKGROUND

The observation and management of a subject after a medical, surgical or any health care intervention is challenging and resource intensive. Monitoring patients, or population based general health metrics, is important to avoid preventable complications, to identify general health deterioration, and assist with post intervention recovery.

At present, monitoring and scoring musculoskeletal health is predominantly performed by healthcare staff, or using questionnaires.

Monitoring various aspects of gait quality and quantity is possible in a subject's normal environment where they are carrying out normal activities with little or no constraint. This can involve the monitoring of various physiological parameters during normal daily activities. For example, during monitoring, a subject may be walking, exercising, engaging in a rehabilitation program or working at either sedentary or active tasks.

Devices to monitor human movement are known. These devices typically use at least one accelerometer to monitor posture, gait or both in real time. In some instances, a wearable monitoring device is attached to a subject, for example by a belt and is aligned to the subjects midsagittal plane.

Other technology uses a pressure-sensing mat, or “walkway,” to measure the relative arrangements of the footfalls as a person walks across the mat, in conjunction with software to process the footfalls to derive certain spatiotemporal gait parameters, such as, e.g., stride length. While this system constitutes the current “gold standard” for gait measurements, it fails to capture, by its nature, real-life walking metrics as seen in day-to-day living. An alternative approach utilizes marker-based motion capture in conjunction with a biomechanical model to derive kinematic parameters. This system is also impractical in a healthcare setting.

Any score of walking performance needs to differentiate the aspects of gait that are problematic for that pathology. For example, patients with lumbar spinal stenosis are known to walk slower, with smaller steps, and with a stooped posture (secondary to neurogenic claudication), whilst patients with multiple sclerosis suffer from uncoordinated movements that manifest as increased walking asymmetry and variability. Other illnesses with a significant psychological or physiological burden, such as depression and cancer, have also been shown to have impaired walking, with reduced walking speed.

There is a need for improved systems and methods to provide robust and reliable quantitative scoring of walking performance, that is relevant to the health status of the patient, based on real-life mobility, using gait metrics to provide useful clinical information on disability status and decline/recovery following any intervention.

### SUMMARY

In a first aspect there is provided a system for determining a quantitative gait score, the system comprising:
a) an accelerometer configured to output signals indicative of movement of the subject along one or any combination of an x-axis, a y-axis, and a z-axis
b) a magnetometer configured to output signals indicative of variations in position of the subject in a space defined by the x-axis, the y-axis, and the z-axis; and
c) a gyroscope configured to output signals indicative of angular velocity of the subject around one or any combination of the x-axis, the y-axis, and the z-axis;
d) a processor configured to receive the output signals and analyse the signals to determine the quantitative gait score for the subject from any combination of the gait metrics:
   i. daily step count;
   ii. step cadence
   iii. step time;
   iv. step time asymmetry;
   v. step length;
   vi. stride length;
   vii. step length asymmetry;
   viii. single support time variability;
   ix. walking orientation randomness metric (WORM Score); and
   x. gait velocity or gait velocity variation;
wherein the x-axis is a horizontal axis to the ground directed forward of the subject's body; the y-axis being a horizontal axis to the ground directed laterally of the subject's body; and the z-axis is vertical axis to ground.

In one embodiment the accelerometer, magnetometer, gyroscope, and optionally the processor are in a sensor unit adapted to be disposed on the subject. The processor may be configured to determine the quantitative gait score from any two or more gait metrics i-x.

In one embodiment the quantitative gait score is at east one of:
i. SMoS calculated from gait velocity and daily step count;
ii. iMoS calculated from step cadence and stride length; and
iii. GSi calculated from gait velocity or gait velocity variation, step length asymmetry and step time asymmetry;
iv. Combined Mobility Score calculated from
   a. daily step count;
   b. one or more of gait velocity, step length, stride length, and cadence
   c. one or more of step length asymmetry, step time asymmetry, single support time variability; and
   d. walking orientation randomness metric (WORM Score);

In a second aspect there is provided a method for determining a quantitative gait score for a subject the method comprising
a) obtaining the gait velocity and daily step count for the subject, assigning a first numerical value to the walking speed, assigning a second numerical value to the daily step count and summing the first and second numerical values to provide the a quantitative gait score;
   wherein
      if the walking speed is above a threshold walking speed the first numerical value is a first maximum numerical value; or
      if the walking speed is below the threshold walking speed, the walking speed is divided by the threshold walking speed to provide a product, the product is multiplied by the maximum numerical value to provide the first numerical value;
   and wherein
      if the daily step count is above a threshold step count the second numerical value is a second maximum numerical value; or
      if the daily step count is below the threshold daily step count, the daily step count is divided by the threshold daily step count to provide a second product and the second product is multiplied by the second maximum numerical value to provide the second numerical value;
or
b) obtaining the step cadence and stride length for the subject and assigning a first numerical value to the cadence, assigning a second numerical value to the stride length and summing the first and second numerical values to provide the quantitative gait score;
   wherein
      if the step cadence is below a first threshold cadence the first numerical value is a first minimum numerical value; or
      if the step cadence is above a second threshold cadence the first numerical value is a first maximum numerical value; or
      if the step cadence is between the first and second threshold cadences, the first threshold step cadence is subtracted from the step and the difference between the step cadence and the first threshold step cadence is the first numerical value;
   and wherein
      if the stride length is below a first threshold stride length the second numerical value is a second minimum numerical value; or
      if the stride length is above a second threshold stride length the second numerical value is a second maximum numerical value; or
      if the stride length is between the first and second threshold stride lengths, the second numerical value is calculated by multiplying the stride length by a value equal to the value of the second threshold stride length to provide a product and then subtracting half of the value from the product to provide the second numerical value;
or
c) obtaining the gait velocity, step length asymmetry and step time asymmetry for the subject; assigning a score to each of the gait velocity, step length asymmetry and step time asymmetry, summing the scores

The quantitative gait score may be at east one of:
i. SMoS calculated from gait velocity and daily step count;
ii. iMoS calculated from step cadence and stride length; and
iii. GSi calculated from gait velocity or gait velocity variation, step length asymmetry and step time asymmetry;
iv. Combined Mobility Score calculated from
   a. daily step count;
   b. one or more of gait velocity, step length, stride length, and cadence
   c. one or more of step length asymmetry, step time asymmetry, single support time variability; and
   d. walking orientation randomness metric (WORM Score);

The quantitative gait score my be SMoS calculated from the sum of gait velocity and step length. If the SMoS for the subject is 1.4 or less the subject has a high fall risk. If the SMoS for the subject is 1.5 or more the subject has a low fall risk.

The quantitative gait score may be iMOS calculated from sum of cadence and stride length. If the iMoS for the subject is 95 or less the subject has a high fall risk. If the iMoS for the subject is 100 or more the subject has a low fall risk.

In one embodiment the quantitative gait score may be GSi calculated from gait velocity or gait velocity variation, step length asymmetry and step time asymmetry. If the GSi for the subject is 0.25 or more the subject has a high fall risk. If the GSi for the subject is 0.2 or less the subject has a low fall risk.

In one embodiment the quantitative gait score is the Combined Mobility Score calculated by
a) obtaining gait metrics in gait categories i-iv:
   i. daily step count;
   ii. one or more of gait velocity, step length, stride length, and cadence
   iii. one or more of step length asymmetry, step time asymmetry, single support time variability; and
   iv. walking orientation randomness metric (WORM Score);
b) assigning a numerical weighting to each gait category; and
c) summing the numerical weightings for the gait categories

If the WORM Score is above a threshold value the numerical weighting assigned to it is not more than 5, 10, 15, or 20 percent of the total of the numerical weightings applied to all the gait categories

If the WORM Score is at or below a threshold value the numerical weighting assigned to it is 20, 25, 20, 35, 40, 45 or 50 percent of the total of the numerical weightings applied to all the gait categories.

### Definitions

As used herein the term ‘IMU’ refers to an Inertial Measurement Unit.

The term ‘AP’ refers to Antero-Posterior.

The term ‘ML’ refers to Medio-Lateral.

The term ‘MEMS’ refers to Micro Electro Mechanical Sensors.

Throughout this specification, unless the context clearly requires otherwise, the word ‘comprise’, or variations such as ‘comprises’ or ‘comprising’, will be understood to imply the inclusion of a stated element, integer or step, or group of elements, integers or steps, but not the exclusion of any other element, integer or step, or group of elements, integers or steps.

Throughout this specification, the term ‘consisting of’ means consisting only of.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification the terms ‘a’ and ‘an’ are used to refer to one or more than one (ie, at least one) of the grammatical object of the article. By way of example, reference to ‘an element’ means one element, or more than one element.

In the context of the present specification the term ‘about’ means that reference to a figure or value is not to be taken as an absolute figure or value but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features and compounds.

In order that the present technology may be more clearly understood, preferred embodiments will be described with reference to the following drawings and examples.

### BRIEF DESCRIPTION OF THE DRAWINGS

Embodiments of the systems and methods are described with reference to the following drawings.

FIG. 1 is a schematic diagram showing preferred locations of the wearable sensor device. Locations may be anterior or posterior.

FIG. 2 is an illustration of the metrics used to calculate the CMoS.

FIG. 3 is a summary of data collection, processing, and outputs from the MetaMotionC sensor and data processing for gait analysis used in this study. FIG. 2a. First output is a .html file which documents the vertical acceleration measured by the sensor (y-axis) against time (x-axis) during the walk done by the participant. Green circles represent the initial foot contact with the ground, usually the ‘heel strike’ phase of gait and orange circles represent the final foot contact with the ground, usually the ‘toe-off’ phase of gait. FIG. 2b. The data processing uses the gait cycle events detected in image A. to identify when a gait cycle begins and ends, and thus creates a .csv file with the values of each gait parameter displayed per gait cycle and for the bout overall. MMC=MetaMotionC sensor from Mbient Labs (or any other 9-axis accelerometer), used to measure gait in the present study.

FIG. 4 is a series of visual representations of the CMOS for normal subjects (FIG. 3A), subjects with a mild impairment of general mobility (FIG. 3B) and subjects with a severe impairment of general mobility (FIG. 3C).

FIG. 5 is a receiver operating characteristic curve (ROC curve) indicating that the Combined Mobility Score can distinguish between healthy and impaired walking.

FIG. 6 is a visual representation of Combined Mobility Score classification system.

### DESCRIPTION OF EMBODIMENTS

The present invention is directed to systems and methods for determining a Walking performance score/s from various gait metrics. The technology uses non-invasive systems and methods to assess and assist with data collection which in turn informs the suitability or necessity for the use of a walking aid such as a walking stick or walking frame; and the suitability of a patient to be discharged from a hospital or other healthcare facility.

The technology is useful for assessing the level of physical disability in the context of recovery from a surgical or medical intervention, particularly in monitoring the effectiveness of an ambulation protocol which can improve patient outcomes but is often overlooked by healthcare staff who have competing clinical duties.

Two-thirds of the body mass is located two-thirds of the height above the ground. To move forward during walking, the center of gravity (upper body) is accelerated forwards of the base of support (lower body). With each step, this center of gravity also oscillates laterally along the line of motion.

Organization of the orientational senses is understood to be an adaptive hierarchical system. There are two main reference frames for the sensory representation of the body posture with respect to space. On a lower level, a weighted combination of orientational inputs directly mediates the activity of postural muscles and mainly controls the horizontal centre of gravity (COG) position. On a higher level, vestibular inputs provide the orientational reference, against which conflicts in support surface and visual orientation are identified and the combination of inputs adapted to the task conditions. For postural stability, the information from the lower level must be coherent with the inertial-gravitational reference of the higher level, and any conflicting orientation inputs must be quickly suppressed in favour of those congruent with the internal reference. Thus, in adults, the sensory organizational process is context specific due to the rapid weighting and re-weighting of sensory inputs to/from the lower level by the higher-level adaptive process.

The systems and methods disclosed herein comprise one or more Inertial Measurement Units (IMUs), commonly known as ‘wearable devices’ or ‘wearables’ which contain various microelectromechanical sensors (MEMS) including accelerometers, gyroscopes and magnetometers. IMUs and are an alternative to the existing methods of gait assessment in laboratory based clinical settings. Wearables can accurately measure numerous gait metrics including gait velocity, stride length, cadence, and step count. Accordingly, the systems and methods disclosed herein can be used to monitor a subject's recovery from surgery or other treatment, to monitor the healing process, or to monitor or verify the extent of the subject's activity, or any combination of these purposes. In some embodiments the systems and devices described herein utilise various gait metrics to determine the SMoS™: “Simplified Mobility Score”. iMoS™: “Immediate Mobility Score”. GSi™: “Gait Stability Index”. In some embodiments the systems and devices described herein utilise various gait metrics to determine the CMOS™: “Combined Mobility Score”

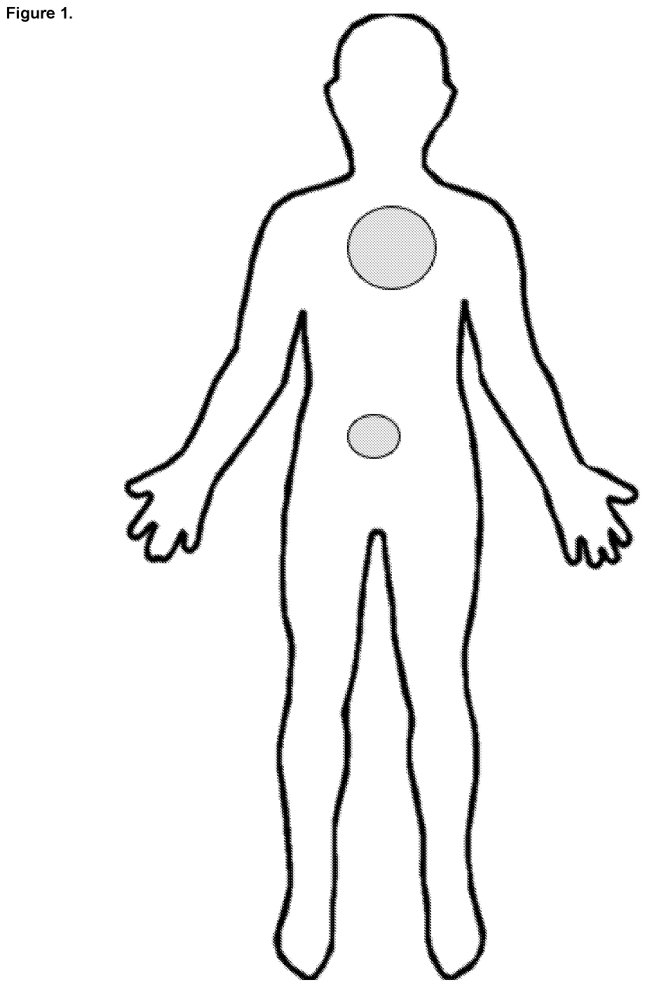

As shown in FIG. 1, the systems and methods use a sensor device placed on a subject's chest, low back, belt line or the like. The sensor may be anterior or posterior. The system includes one or more sensor devices that communicate with a processor that can produce information, based on the sensor readings and data, to facilitate the patient or another user, such as a clinician, doctor, hospital, carer, or other appropriate person, monitor the subject.

The system includes a wearable device with one or more sensors, such as accelerometers. For example, the wearable device may include one or more sensors and may be applied to the skin of a subject. In at least some embodiments, the one or more sensors communicate with a processor. The processor may be in the wearable device or may be remote from it. In some embodiments the sensor device also includes a display. In some embodiments, the processor, the sensors, or both communicate with a display device, such as a mobile phone, tablet, or computer.

The system includes a processor, and one or more sensors in a wearable device. Optionally, the system includes a display device, such as a mobile phone, tablet, or computer that may comprise the processor and may be used to process and/or display information obtained or derived from the sensor device.

In at least some embodiments, the one or more sensors and, preferably, the processor (or multiple processors) are provided in a sensor device that is adapted to be applied to the skin of the patient, carried on an article of clothing or carried on a sling or harness worn by the patient.

The display device can be any suitable device such as a computer (for example, a notebook or laptop computer, a mobile medical station or computer, a server, a mainframe computer, or a desktop computer), mobile devices (for example, a smartphone, smartwatch, or a tablet), or any other suitable device. In some embodiments, the display device can be incorporated into a medical station or system.

In some embodiments the display device is configured to communicate with one or more other devices and can for example alert a subject's clinician, career or other designator person or service.

In one embodiment of the sensor device, the display device, or both have the ability to process data and comprise a memory, a display, and are adapted to receive an input via an input device. In some embodiments these components can be carried by the user (for example if they are part of the sensor device).

The processor is configured to execute instructions provided to the processor. Such instructions can include any of the steps of methods or processes described herein. Any suitable memory can be used for the sensor and display devices. The memory may be any computer-readable storage media such as, nonvolatile, non-transitory, removable, and non-removable computer-readable media implemented in any method or technology for storage of information, such as computer readable instructions, data structures, program modules, or other data.

Communication methods provide another type of computer readable media, e.g. communication media. Communication media typically embodies computer-readable instructions, data structures, program modules, or other data in a modulated data signal such as a carrier wave, data signal, or other transport mechanism and includes any information delivery media. By way of example, communication media includes wired media such as twisted pair, coaxial cable, fiber optics, wave guides, and other wired media and wireless media such as acoustic, RF, infrared, Bluetooth', near field communication, and other wireless media.

The display can be any suitable display such as a monitor, screen, display, or the like, and can include a printer.

The input device can be, for example, a keyboard, mouse, touch screen, track ball, joystick, voice recognition system, camera, microphone, or any device known in the art to provide input directly or indirectly to a processor.

Any suitable type of sensor can be used including, but not limited to, accelerometers, magnetometers, gyroscopes, proximity sensors, infrared sensors, ultrasound sensors, thermistors or other temperature sensors, cameras, piezoelectric or other pressure sensors, sonar sensors, external fluid sensor, skin discoloration sensor, pH sensor, and microphones or any combination thereof.

In at least some embodiments, the system includes at least one, two, three, four, five, six, or more different types of sensors. The system may include at least one, two, three, four, five, six, eight, ten, or more sensors. The sensors may be present in a single sensor device or in multiple sensor devices adapted to be applied to different areas of the subject.

The one or more sensor devices can be used to measure, monitor, or otherwise observe a subjects gait metrics and therefore their physical activity or health; recovery from surgery or other treatment; rehabilitation program, or any combination thereof.

Information sufficient to calculate one or more of the following can be obtained by the sensors: gait velocity, step length, stride length, step cadence, step time, step time asymmetry, step length asymmetry, gait velocity variation, step time variation, and step length variation. Other examples of observations or measurements that can be made or interpreted using one or more of the sensors include activity, temperature of skin, pulse or pulse profile or heart rate recovery time after activity, sleep profile or rest duration. The system can observe or measure one or more of these items or any combination of the items.

The sensor device may be adapted to adhere to the skin or otherwise be held adjacent to the skin of the subject. The sensor device typically includes a housing and an adhesive pad to attach the base to the skin of the subject. Alternatively, the housing may be adapted to attached to an article of clothing. Within the housing the sensor device comprises one or more sensors, a power source, a communications unit, and optionally a processor.

The housing can be made of any suitable material, such as plastic or silicone, and has sufficient flexibility to fit comfortably to or rest adjacent to the subject's skin. In some embodiments the housing is also resistant to water, sweat, and other fluids. In some embodiments the housing is sufficiently water resistant to allow the patient to shower or bathe with the sensor device.

In some embodiments the sensors, power source, communications unit, and processor are contained within the housing. In some embodiments, a portion of one or more of the sensors, such as a temperature, pulse, or pressure sensor, moisture sensor, or strain gage, may protrude through the housing to allow contact of the sensor or part o the sensor with the skin of the patient.

In some embodiments of the sensor device comprises an accelerometer, a gyroscope and a magnetometer. The accelerometer, gyroscope and magnetometer can be used to measure gait metrics as noted above.

Other suitable sensors include, but are not limited to, a microphone, pulse oximetry sensor, a heart rate monitor, or the like, or any combination thereof. As will be understood, any suitable sensor described above can be included in the sensor unit and any combination of those sensors can be used in the sensor unit.

Power can be provided to the sensors and processor using any suitable power source such as primary cells, coin cell batteries, rechargeable batteries, storage capacitors, other power storage devices, or any combination thereof. In some embodiments, the power is provided by a kinetic energy power to power the components or to or to recharge a battery or other power source coupled to the components. In some embodiments, a wireless power source can be used. In some embodiments the sensor device comprises a charging port for charging the power source. Alternatively or in addition, wireless charging systems and methods can be used.

All of the sensors and the processor may be coupled to the same power source or some of the sensors (or even all of the sensors) and sensor processor may have individual power sources.

In some embodiments, the sensors and processor are continuously active. In other embodiments, the sensors and processor are active intermittently (for example every 0.1, 0.5, 1, 5, 10, 15, or 30 seconds). Optionally, the period may be programmable. In one embodiment the period is altered based on data from one or more of the sensors. In another other embodiment the sensors and processor are activated manually or automatically by the sensor device or display device. In some embodiments the sensors and processor are activated automatically when the sensor device is put into motion.

In some embodiments, each sensor may have different activation schedules (e.g. continuous, intermittent, manual). For example, a temperature sensor may measure temperature periodically, a sensor to measure gait velocity or step asymmetry may be activated automatically when motion is detected.

The processor can be any suitable processor and may include or be coupled to memory for storing data received from the sensor. The processor can be wired or wirelessly coupled to the sensor. In some embodiments, the processor may include analysis algorithms for analyzing or partially analyzing data received from the sensor. In other embodiments, the processor may be used to receive, store, and transmit data received from the sensors.

The communications unit can be any suitable communications arrangement that can transmit information from the processor or sensors to another device (such as the display device) The communications unit can transmit this information by any suitable wired or wireless technique such as Bluetooth, near field communications, WiFi, infrared, radio frequency, acoustic, optical, or by a wired connection through a data port in the sensor device.

The systems and methods can utilise personal characteristics of the subject to assist in determining one or more gait metrics and walking scores. The personal characteristics can include one or any combination of age, gender, height, weight, level of activity, level of mobility, body mass index (BMI), leg length discrepancy, and surgical procedure. In some embodiments, the gait metrics may differ based on the subject's gender, age, or height (or any other personal characteristic or combination of personal characteristics).

In at least some embodiments, the ranges for the different measurements can be modified for age, gender, height, or other personal characteristics, or any combination thereof. An application on the display device may provide information regarding the measurements (for example, lists of the measurements, graphs of the measurements, averages or daily numbers for the measurements or the like or any combination thereof), as well as any of the metrics described above such as the SMoS score. The application may allow a user to access to some or all profile details and may permit access to sensor unit set-up and calibration applications or protocols.

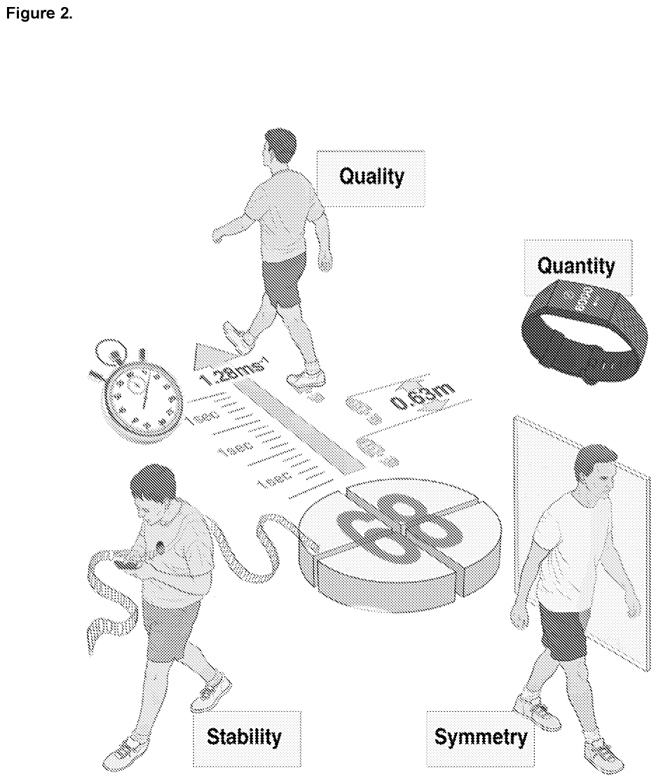

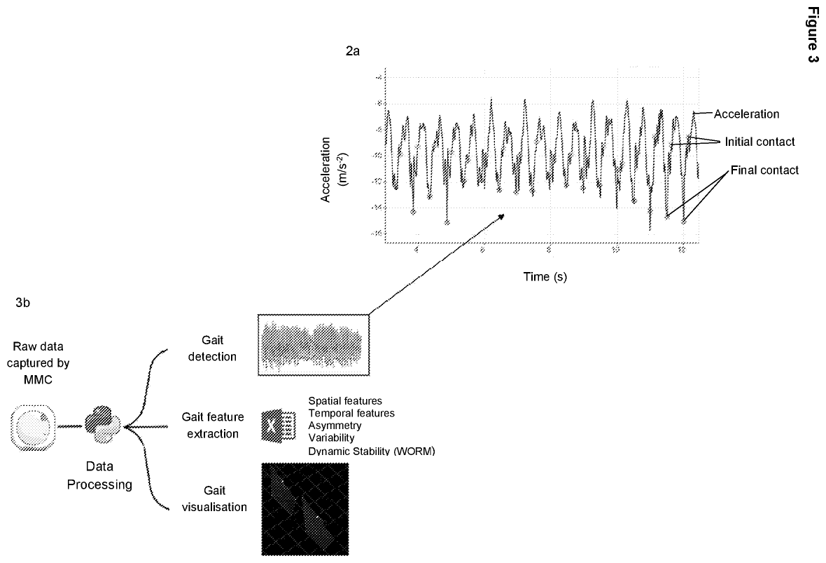

In some embodiments commercially available sensors and software packages such as the MetaMotionC sensor (or any other 9-axis accelerometer) may be used for gait analysis. However, a skilled person will be able to create suitable code for data collection from the sensor (for example a 9-axis accelerometer), data processing, and outputs for gait analysis. The first output from the IMU may be a .html file which documents the vertical acceleration measured by the sensor (y-axis) against time (x-axis) during the walk done by the subject. Green circles represent the initial foot contact with the ground, usually the ‘heel strike’ phase of gait and orange circles represent the final foot contact with the ground, usually the ‘toe-off’ phase of gait. FIG. 2b. The program uses the gait cycle events detected in image a to identify when gait cycles begin and end, and thus creates a .csv file with the values of each gait parameter displayed per gait cycle and for the bout overall, these data can be use to calculate gait metrics (see below). Additionally, a .c3d file is created which can be viewed using any suitable viewer known in the art, for example Mokka (an open source platform), to create a visual recreation of the gait using the accelerometry data.

Wearable sensors can sample data at a range of rates. For example, as exemplified herein the sensor at a rate of 100 hz. However, it is envisaged that sample rates from around 20 Hz to 600 Hz, for example suitable sampling rates may be 20, 30, 40, 50, 60, 70, 80, 90, 100, 125, 150, 175, 200, 225, 250, 275, 300, 325, 350, 375, 400, 425, 450, 475, 500, 525, 550, 575, or 600 Hz. Sampling rates in excess of 600 Hz are also compatible with the methods and systems described herein.

### Gait Metrics

The systems and methods described herein utilise one or more of the following gait metrics. While the gait metrics can be calculated using any known methods, the exemplary methods below assume that n steps were taken over the entire bout, or for the walking orientation randomness metric calculation, that the bout was n meters long.

The CMOS is a scored measure of walking performance based on objective data captures. In one embodiment, the CMOS ranges from 0 (poor performance) to 100 (excellent performance) utilizing the following gait categories. It is envisaged that any scoring system may be used.
i. Quantity: Average daily step count
ii. Quality: one or more of gait velocity, step length, stride length, and cadence
iii. Consistency: one or more of step length asymmetry, step time asymmetry, single support time variability
iv. Stability: WORM Score (see below)

GV=Gait Velocity; ST=Step time; SL=Stride length; STA=Step time asymmetry; SLA=Step length asymmetry. Step length may also be used, step length is half the stride length.

$$GV = \frac{Total distance}{Total time} = \frac{average SL}{average ST} = \frac{{\sum }_{i = 1}^{n} GV of {step}_{i}}{2 n} \qquad Equation 1$$

$$ST = \frac{{\sum }_{i = 1}^{n} {steptime}_{i}}{2 n} \qquad Equation 2$$

$$SL = \frac{{\sum }_{i = 1}^{n} = {steplen}_{i}}{2 n} \qquad Equation 3$$

$$STA = \frac{{\sum }_{i = 2}^{n} ( {steptime}_{i} - step {time}_{i - 1} )}{2 n} \qquad Equation 4$$

$$SLA = \frac{{\sum }_{i = 2}^{n} ( {steplength}_{i} - ste {length}_{i - 1} )}{2 n} \qquad Equation 5$$

The studies described herein focus on the application of metrics to score gait.

Transverse truncal motion during walking, is independent of (but correlates with) other balance variables. Laboratory-based posturography measurements of centre of pressure (CP), quantify the (weighted average) vertical force vector exerted by a subject on a force platform. These studies have identified anteroposterior (AP) and mediolateral (ML) displacements, velocity and total path lengths of CP as the variables differentiating fallers from non-fallers. However, these CP measurements almost completely ignore the behaviour of the upper half of the body. Moreover, as CP measurements are taken whilst standing unperturbed (static) or whilst moving (dynamic), they assess postural control rather than stability or balance of walking.

Conversely, centre of mass (COM) movement more closely represents balance. Recent, wearable accelerometery studies of walking (dynamic) balance have assessed ambulatory CM movement in fallers as a measure of dynamic balance. These studies typically considered amplitudes and variability of CM motion along (mediolateral or vertical) axial planes, with the sensor-placement most commonly at the approximate centre of mass: along the lumbar vertebrae.

Previous studies on centre of gravity (CG) motion calculated from the ‘mechanical work’ of individual joint segments, established pathological gait patterns to involve greater mechanical energy expenditure. These mechanical inefficiencies likely arise due to compensatory inclinations of the trunk and motions of the upper limbs that seek to offset pathological lower limb biomechanics (and prevent falls).

### Walking Orientation Randomness Metric (WORM) Score

In one embodiment Let Li be the total length of the path taken by point X relative to point O in the horizontal plane during meter i of a walking bout of n meters.

$$W O R M = \frac{{\sum }_{i = 1}^{n} L i}{n} \qquad Equation 6$$

During walking, the summative motions of individual joint segments accelerate the trunk forwards of the base of support. With each step, the trunk also oscillates laterally along a line of motion situated at the medial borders of either feet.

To reflect the aforementioned ‘inverted pendulum motion’ of the trunk, the WORM score measures the ‘wobble’ of the upper body as measured from a single-point IMU with a chest-based attachment as shown in FIG. 1. In this illustration, the body frame B, has its x-axis aligned with the initial direction of walk (antero-posterior plane), z-axis aligned with the direction of measured acceleration due to gravity (superio-inferior plane), and y-axis calculated as the cross product of z and x axis (medio-lateral plane).

In another embodiment to calculate the WORM score, first calculate the point pₜ at time step t from the orientation of the body with respect the world frame, ^{W}Rₜ^{B}. The body orientation, ^{W}R^{B}, is obtained from the orientation measured by the single point IMU, ^{W}R̆ₜ^{S}, adjusted by a fixed sensor-to-body rotational offset, ^{B}R₀^{S}, as shown in Equation 15. The sensor-to-body offset, ^{B}R₀^{S}, was calculated by assuming an upright pose (i.e., ^{W}R₀^{B}=I_{3×3}) at t=0 as shown in Equation 8. Finally, point pₜ, which is effectively the x and y coordinates of the body z axis with centre at the origin, is calculated using Equation 9.

$${}^{W} {R}_{t}^{B} = {}^{W} {{R}^{⌣}}_{t}^{S} {( {}^{B} {R}_{0}^{S} )}^{T} \qquad Equation 7$$

$${}^{B} {R}_{0}^{S} = {}^{W} {{R}^{⌣}}_{0}^{S} \qquad Equation 8$$

$${p}_{t} = [ \begin{matrix}1 & 0 & 0 \\ 0 & 1 & 0\end{matrix} ] {}^{W} {R}_{t}^{B} [ \begin{matrix}0 \\ 0 \\ 1\end{matrix} ] \qquad Equation 9$$

In this embodiment and referring to FIG. 3, the WORM score (or WORMdist) is the distance travelled in the transverse plane travel by pₜ.

In some embodiments the WORM Score is used as a standardised method of assessing walking stability and balance. As described an exemplary embodiment has validated its utility to distinguish fallers from non-fallers within a sample population of 32 participants and demonstrates that the WORM Score identifies an 8-fold increase in fallers compared to non-fallers. Fallers had significant differences in spatiotemporal parameters of gait with lower gait velocity, step length and cadence despite greater step time. Fallers also walked with greater asymmetry (in step time but not step length) and variation (in gait velocity, step length and step time). In terms of falls classification the WORM score provides good discriminative accuracy (AUC>0.90) with gait velocity, step time, step time asymmetry, gait velocity variation.

The study described herein focuses on the application of wearable-sensors for fall risk assessment and through wearable accelerometry the inventors have identified the relevant gait variables with high predictive classification accuracy in distinguishing fallers from non-fallers.

Transverse truncal motion during walking, is independent of (but correlates with) other balance variables. Laboratory-based posturography measurements of centre of pressure (CP), quantify the (weighted average) vertical force vector exerted by a subject on a force platform. These studies have identified anteroposterior (AP) and mediolateral (ML) displacements, velocity and total path lengths of CP as the variables differentiating fallers from non-fallers. However, these CP measurements almost completely ignore the behaviour of the upper half of the body. Moreover, as CP measurements are taken whilst standing unperturbed (static) or whilst moving (dynamic), they assess postural control rather than stability or balance of walking.

Conversely, centre of mass (COM) movement more closely represents balance. Recent, wearable accelerometery studies of walking (dynamic) balance have assessed ambulatory CM movement in fallers as a measure of dynamic balance. These studies typically considered amplitudes and variability of CM motion along (mediolateral or vertical) axial planes, with the sensor-placement most commonly at the approximate centre of mass: along the lumbar vertebrae.

Previous studies on centre of gravity (CG) motion calculated from the ‘mechanical work’ of individual joint segments, established pathological gait patterns to involve greater mechanical energy expenditure. These mechanical inefficiencies likely arise due to compensatory inclinations of the trunk and motions of the upper limbs that seek to offset pathological lower limb biomechanics (and prevent falls). Thus, a greater WORM score (truncal motion) identifies these falls-risk participants with compensatory gait alterations.

The findings of significant differences in a range of gait variables between fallers and non-fallers reinforces the potential use of gait analysis in the detection, preferably early detection, of gait and balance impairments.

The methods described herein provide objective, unsupervised and unobtrusive method of point-of-care testing to assess walking stability and balance in clinical settings and/or home environments. The ‘WORM Score’ provides clinicians, patients, and carers with a quantification of walking instability serving as an accurate, and sensitive biomarker for monitoring functional balance and falls-risk. Further, such identification of gait and balance deficits can prompt timely intervention before a fall occurs and, therefore improve quality of life and avert the need for an additional, or higher-level intervention in the future.

Traditionally, objective monitoring of gait and balance with laboratory-based techniques such as optoelectronic stereophotogrammetry require extensive resources (equipment, trained personnel), and can be time-consuming.

The WORM score is a quantitative measure of walking instability for long-term monitoring and assessment. Accordingly, it can be used in multiple scenarios, including care of falls-prone patients, determining suitability for walking aids, physical therapy, home modifications, altering medication regiments (for geriatric patients) or dose alterations (for instance in Parkinson's disease).

Objective and quantitative falls-risk assessment in the clinical setting via the WORM score also has post-intervention applications in assessing suitability for safe discharge of a patient. Regardless of the intervention (medical or surgical), or presenting complaint (head injury, trauma, or acute/chronic illness) assessment of walking stability prior to discharge benefits patients and health-care systems alike, by minimising post-discharge falls in the community. The WORM score can also inform home care and rehabilitation.

Suitability for falls-preventive interventions (such as walking aids) can also be assessed by the objective and quantitative assessment of walking stability provided by the WORM Score or by a combination of gait metrics. With either age or injury, a point in time may arise when an individual requires mobility assistance. This may include a walking stick, or a walking frame. WORM scores for non-fallers versus fallers are defined herein however the intermediate scores between these 2 points provide an indication that the patient would benefit from walking aid.

In one embodiment, a WORM score for a patient of 0.2 or less is indicative of a minimal risk of falling.

A WORM score for a patient of 0.2-0.6 indicative of a low risk of falling.

Patients with a minimal or low risk of falling are capable of walking unaided.

A WORM score for a patient of 0.6-1.2 indicative of a medium risk of falling

In one embodiment a WORM score for a patient of more than 1.2 is indicative of a high risk of falling.

Advantageously, the WORM score can be calculated from as few as 6 gait cycles.

Accordingly, this allows for the systems described herein to include a means to alert the patient, their physician or carer of a falls risk.

In other embodiments two or more of the gait metrics described by equations 1-13, or by any other means known on the art, can be used to determine a fall risk for a subject. In these embodiments the sum of the gait metrics is indicative of fall risk. For example if the sum of the gait metrics meets or exceeds a predetermined threshold value the subject is at risk of falling. In some embodiments if the sum of the gait metrics meets or falls below a predetermined threshold value the subject is at risk of falling.

For example if the sum of gait velocity and step length for a subject is 1.4 or less the subject is physically unstable and has a high fall risk. If the sum of gait velocity and step length for a subject is 1.5 or more the subject is physically stable and has a low fall risk.

In another embodiment if the sum of cadence and step time for a subject is 95 or less the subject has a high fall risk. If the sum of gait velocity and step length for a subject is 100 or more the subject has a low fall risk.

In another embodiment if the sum of step time asymmetry and step length asymmetry for a subject is 0.25 or more the subject has a high fall risk. If the sum of step time asymmetry and step length asymmetry 0.2 or less the subject has a low fall risk.

In a further embodiment if the sum of gait velocity variation, step time variation and step length variation for a subject is 0.25 or more the subject has a high fall risk. If the sum of gait velocity variation, step time variation and step length variation is 0.2 or less the subject has a low fall risk.

### CMOS Scoring Algorithm

To calculate the CMOS for a subject, each metric is assigned a score out of 100 (or some other number). A score out of 100 is convenient as the score is equivalent to the percentile of the value of the metric relative to a database of healthy control subjects. The overall score of the gait category is the average score of its constituent metrics, or any combination of its constituent metrics. If the WORM Score is not in the bottom percentile, then the weightings of each category are shown in Table 1. That is if the WORM Score is above a threshold (e.g. the bottom percentile) the weighting assigned to it, in this embodiment is 10 (i.e. 10% of the combined weightings). Alternatively the weighting assigned to it may be not more than 5, 10, 15, or 20 percent of the total of all the weightings.

TABLE 1
Category weightings when WORM score
above a predetermined threshold

| Gait category | Weighting |
|---|---|
| Quantity | 40 |
| Daily step count | |
| Quality | 30 |
| Gait velocity | |
| Cadence | |
| Stride length | |
| Consistency | 20 |
| Step length asymmetry | |
| Step time asymmetry | |
| Double step time variation | |
| Stability | 10 |
| Walking orientation Randomness Metric (WORM Score) | |

However, if the subject's WORM score is in the bottom percentile, then the weightings are adjusted to reflect the subject's considerable instability, as shown in Table 2. That is if the WORM Score is at or below a threshold (e.g. the bottom percentile) the weighting assigned to it, in this embodiment is 50 (i.e. 50% of the combined weightings). Alternatively, if the WORM Score is at or below a threshold value the weighting assigned to it is 20, 25, 20, 35, 40, 45 or 50 percent of the total of the numerical weightings applied to all the gait categories. percent of the total of all the weightings.

TABLE 2
Category weightings when WORM score
is below a predetermined threshold

| Gait category | Weighting |
|---|---|
| Quantity | 40*(5/9) |
| Daily step count | |
| Quality | 30*(5/9) |
| Gait velocity | |
| Cadence | |
| Stride length | |
| Consistency | 20*(5/9) |
| Step length asymmetry | |
| Step time asymmetry | |
| Double step time variation | |
| Stability | 50 |
| Walking orientation Randomness | |
| Metric (WORM Score) | |

In one embodiment the resultant total is multiplied by 1.4, with the maximum final CMOS capped at 100.

For each metric, lower cut-off values were well placed at the level of a severe walking impairment (below which further deterioration is not clinically meaningful), while upper cut-off values represented the upper limit of ‘normal’ function (above which further improvement is not clinically meaningful), with cut-off values obtained form large-scale studies known in the art. The CMOS can be used for the comprehensive evaluation of patients in any clinical scenario.

In some embodiments the CMOS is recorded continuously using a wearable device and may be streamed to healthcare providers or carers from any location, allowing for an objective, real-time evaluation of walking performance.

### EXAMPLES

### Example 1: Simplified Mobility Score (SMoS)

Simple scores such as the SMoS, calculated from the gait metrics assists in the rapid identification of individuals, or populations, with declining health, facilitating early intervention, which may delay the typical increased healthcare costs and diminished quality of life associated with ageing and frailty.

The SMoS was tested in a sample of patients with spinal pathologies and compare them to population samples in order to validate the tool as a simple screening tool for deterioration in walking quality. Given that walking quality is diminished in a number of disease states, the experimental group is expected to have lower SMoS scores across age and gender strata than the population norms. SMoS is a simple measure of walking quality that is quickly and easily obtained from data captured by a patient's smart device, thus providing additional clinical information without sacrificing time.

### 1.1 Methods

The Simplified Mobility Score (SMoS™), can be measured using the daily step count and walking speed obtained from smart devices like the Apple iphone, Apple Watch, Android devices or similar products. Both gait velocity and step count are given a score out of 50 (or any other number a user may choose) using a linear calculation with an upper limit or threshold, calculated as a percentage of the upper limit, and multiplied by 50, which in this case is equal to the threshold (Table 3). The sum of the two scores is the overall SMoS score. The upper limit or threshold of 50 was chosen to delineate those with functional disability from those without any limitation to their daily physical and functional activities who would be expected to have no negative outcomes resulting from impacted gait.

TABLE 3
Calculation of the SMoS based on the
primary gait metrics of WS and DSC

| Walking Speed (WS) | | Daily Step count (DSC) | |
|---|---|---|---|
| WS (v) | Points (A) | DSC | Points (B) |
| v < 1.35 m/s | (v/1.35 m/s) × 50 | DSC < 10,000 | (DSC/10,000) × 50 |
| v > 1.35 m/s | 50 | DSC > 10,000 | 50 |
| sMos = A + B | | | |

The present study was a retrospective observational study using a database of 450 consecutive patients (aged 30 and over) presenting for the first time to a single spinal neurosurgery clinic with pain and/or sensorimotor deficits. Each patient was consented to the study and completed a questionnaire with demographic information and disability scores (ODI, NDI, VAS). Patients were taken for a timed walk along an unobstructed pathway over a self-selected distance (30, 60, 120 or 200 meters) to measure gait velocity. DSC was obtained from their smart device based on the data over the last month of tracking. Gait data was available for 182 patients. Patients were excluded if they were unable to walk independently without a device or human assistant, and if they were under the age of 18.

The sample data for walking speed and daily step count were compared to expected population values obtained from large population studies measuring walking speed (n=23,111—Bohannon et al. Physiotherapy. 2011; 97 (3): 182-9) and daily step count (n=717,527—Althoff T, et al. Nature. 2017; 547 (7663) 336-9). Two-tailed z-tests were used to test for statistically significant differences between the sample data and the population values, with significance defined at p<0.05.

Subgroup analyses were also performed by age group and gender. A two-tailed independent sample z-test was performed to determine whether there was a statistically significant difference in the mean SMoS score between patients that underwent surgery and patients that did not. The Shapiro-Wilk test for normality was performed to maintain the assumptions of the chosen statistical test. Pearson's correlation analysis was used to determine the association of SMoS with ODI, NDI and VAS scores. Data was collected and processed using IBM SPSS Statistics, version 26.

### 1.2 Results

Ninety-two women and 90 men were eligible for analysis and calculation of their SMoS. The mean age was 56 years (range 20-88), and 38 (21%) had surgical intervention for their spinal pathology within the study period; the average time until surgery was 2 months. The mean ODI was 40 (range 0-98), NDI was 26 (range 0-68) and VAS was 7 (range 0-10). The population mean of SMoS within age, gender and pathological subgroups are displayed in Table 4.

TABLE 4
Statistical analyses for the reduction in SMoS in spine patients compared
to expected population values, separated by age group and gender.

| | Population | Sample | | Difference | |
|---|---|---|---|---|---|
| Age group | Mean (± SD) | Mean (± SE) | n | Mean difference (95% CI) | P |
| Total | 74.3 ± 3.63 | 58.7 ± 1.68 | 182 | −15.6 (−18.90 to −12.30) | <.001 |
| Male | 77.2 ± 9.75 | 60.0 ± 2.42 | 90 | −17.2 (−21.95 to −12.45) | <.001 |
| Female | 69.7 ± 8.73 | 57.0 ± 2.40 | 92 | −12.7 (−17.40 to −8.00) | <.001 |
| 30-39 | 75.9 ± 3.90 | 66.4 ± 4.03 | 32 | −9.5 (−17.40 to −1.60) | <.001 |
| Male | 78.6 ± 9.59 | 64.1 ± 5.39 | 20 | −14.5 (−25.06 to −3.94) | <.001 |
| Female | 71.2 ± 9.20 | 70.2 ± 6.03 | 12 | −1.1 (−12.83 to 10.83) | 0.689 |
| 40-49 | 75.1 ± 3.31 | 63.4 ± 3.84 | 41 | −11.7 (−19.23 to −4.17) | <.001 |
| Male | 77.5 ± 9.77 | 59.2 ± 5.39 | 24 | −18.3 (−28.86 to −7.74) | <.001 |
| Female | 70.9 ± 8.20 | 68.7 ± 5.09 | 17 | −2.2 (−12.18 to 7.78) | 0.258 |
| 50-59 | 73.7 ± 4.39 | 68.8 ± 4.35 | 30 | −4.8 (−13.42 to 3.62) | 0.348 |
| Male | 76.5 ± 9.56 | 75.2 ± 6.09 | 14 | −1.3 (−13.24 to 10.64) | 0.465 |
| Female | 68.6 ± 9.30 | 63.3 ± 5.9 | 16 | −5.3 (−16.84 to 6.26) | 0.023 |
| 60-69 | 68.5 ± 3.49 | 57.0 ± 3.2 | 36 | −11.5 (−17.77 to −5.23) | <.001 |
| Male | 74.2 ± 9.71 | 54.6 ± 4.41 | 17 | −19.6 (−28.25 to −10.95) | <.001 |
| Female | 64.0 ± 8.50 | 49.7 ± 4.66 | 19 | −14.3 (−23.43 to −5.17) | <.001 |
| 70+ | 60.4 ± 3.53 | 46.7 ± 2.42 | 43 | −13.7 (−18.45 to −8.95) | <.001 |
| Male | 65.0 ± 10.1 | 49.9 ± 3.25 | 16 | −15.1 (−21.47 to −8.73) | <.001 |
| Female | 55.5 ± 8.3 | 44.9 ± 3.33 | 27 | −10.6 (−17.13 to −4.07) | <.001 |

n number of individuals

The mean SMoS for non-operative patients was 62.1 (SD=22.97) and 50.2 (SD=21.25) for operative patients. Operative patients had a mean SMoS that was 11.9 points lower than non-operative patients (p<0.0033, 95% CI −19.88 to −4.018).

Pearson's correlation coefficient between SMoS and ODI, VAS and NDI were −0.570 (p<0.001, r2=0.3252), −0.561 (p<0.001, r2=0.314) and −0.037 (p=0.855, r2=0.001), respectively. This indicates a moderate negative correlation with ODI and VAS, but no correlation with NDI.

### 1.3 Discussion

New patients presenting to a spinal surgery clinic displayed statistically significantly lower SMoS than expected from large population data samples and remained true when subjects were age and gender-matched to population data. Subgroup analysis revealed that patients who progressed to surgical intervention in the following 3 years had significantly worse SMoS than non-operative patients. This implies that the SMoS has differentiating power between patients with advancing disease severity, from none, to mild (not requiring intervention) to severe (requiring intervention) within the age and gender strata. Given the ambiguity of when to surgically intervene in conditions such as spinal stenosis, the SMoS could become a useful and quick tool in the future which could provide additional information to aid this decision.

Patients with high SMoS also had much lower physical disability according to well-established disability scores such as ODI and VAS, while low SMoS scores predicted high disability on the subjective measures of ODI and VAS. This is suggestive of the validity of the SMoS as a marker of physical disability and is in accordance with existing literature which suggests both walking speed and daily activity levels are reduced in the presence of diseases that affect the neurological and musculoskeletal systems [1, 6, 21]. While the SMoS should not replace these measures of disability, it can act as a useful adjunct to more holistically evaluate these patients.

Musculoskeletal disorders outside of the spine involved in walking, such as knee and hip osteoarthritis, also result in poor kinematic parameters including reduction in walking speed [22, 23]. Given the known association of walking quality with functional disability, and the additional association of walking quality with disease-specific disability scores in the present study, the SMoS may also be used to guide functional intervention by occupational and physiotherapy. The SMoS may also be used in the long-term monitoring of patient functional and disease status with a lower threshold for intervention. Given its ease of use and almost universal availability, there are very few barriers to the implementation of the SMoS in spinal surgical practice. Future studies in the fields of geriatrics, orthopaedics, mental health and other non-surgical neurological disorders will enhance the uptake of the SMoS as a routine practice as a clinical screening tool for both individual and population-based assessment.

The detection and quantification of decline and recovery in physical and mental health status, across a broad range of pathologies, remains a challenge using a simple and single health measure, or score. The SMoS promises to be a tool that relays significant information about the individual that is easy for any health practitioner to understand and will assist with a range of healthcare decisions about a patient, while being easy to collect using readily available devices. Overall, there appears to be no major drawback to implementing the SMoS as a routine aspect of clinical evaluation, especially in gait disorders where it can be used to monitor disease progression and prognosticate.

### Example 2: Immediate Mobility Score (iMoS)

Objective gait analysis is centred around the deconstruction of gait patterns into quantifiable parameters. This typically includes parameters of physical activity, such as step count and distance travelled, alongside spatial and temporal gait parameters, such as walking speed, step length, stride length, step time, and cadence (and their respective derivations of asymmetry and variability) Some methods have also attempted to quantify dynamic postural stability. However, the influx of several complex metrics is difficult to interpret simultaneously, and clinicians may benefit from simple summary scores such as the Immediate Mobility Score (iMoS). The iMoS is a summary score of the metrics stride length and cadence (the inverse of step time). These parameters are amongst the simplest in the field of objective gait analysis and have been investigated across multiple disease settings. They can be computed by single-point wearable sensors—including most smartphones—and manually in a clinical setting using a stopwatch and pedometer. The study described herein is a prospective non-blinded single-centre cohort study comparing the iMoS™ in a sample of patients with walking impairments and a sample of healthy controls.

The iMOS is a scored measure of single time-point walking performance based on objective data capture, ranging from 0 (poor performance) to 100 (excellent performance). It is a summary score encapsulating the simplest, core gait parameters—gait velocity (walking speed, in meters per second), cadence (in steps per minute, reflecting the duration of each step), and stride length (in meters per stride, reflecting the distance covered by each stride, equivalent to 2 steps). Cadence was selected instead of step or stride time as it is easier to manually measure (simply counting the number of steps taken after the passage of one minute). Since gait velocity, cadence, and stride length are mathematically related (using the aforementioned units):

Gait velocity=(Cadence)*(Stride length)/120.

A summary score containing two of these metrics is sufficient to represent all three. As such, the iMoS is derived from the metrics cadence and stride length, with cadence given a weighting of 70% and stride length 30% (Table 5) (with weightings determined by binary logistic regression; see Supporting Information). For each metric, lower cut-off values were placed at the level of a severe walking impairment (below which further deterioration is not clinically meaningful; 50 steps per minute, and 0.5 m per stride), while upper cut-off values represented the upper limit of “normal” function (above which further improvement is not clinically meaningful), with cut-off values obtained from large-scale studies in the literature. The iMoS can be used for the rapid evaluation of patients with a suspected or known walking impairment using either a wearable sensor or manually calculated metrics. The iMoS has the potential to be recorded continuously using a wearable device and streamed to health care providers from a remote location, allowing for an objective, real-time evaluation of walking performance.

TABLE 5
Scoring of iMoS metrics.

| | | |
|---|---|---|
| Metric | Range | Score |

| | | | |
|---|---|---|---|
| Cadence (c) | <50 | steps per minute | 0 |
| | 50-120 | steps per minute | c − 50 |
| | >120 | steps per minute | 70 |
| Stride | <0.5 | m | 0 |
| length (l) | 0.5-1.5 | m | 30/−15 |
| | >1.5 | m | 30 |

| | |
|---|---|
| iMoS ™ total | 100 |

iMoS = Immediate Mobility Score; cadence of 120 steps per minute is considered the upper limit or threshold of normal for healthy adults; stride length of 1.5 m is considered the upper limit or threshold of normal for healthy adults.

### 2.1 Study Participants

A cohort of patients with lumbar spine pathologies, with a principal diagnosis of either lumbar spinal stenosis, lumbar disc herniation, or mechanical low back pain, were recruited. An additional cohort of falls patients who were judged by an experienced geriatrician and neurosurgeon to have a clearly severe walking impairment were also recruited. Healthy control subjects were recruited from the community using verbal outreach. Inclusion and exclusion criteria are provided in Table 6. For each participant, study parameters and risks were discussed, and written consent obtained.

TABLE 6
Eligibility criteria for study cohorts.

| | |
|---|---|
| Healthy | Inclusion criteria |
| controls | Aged greater than 18 |
| | Exclusion criteria |
| | Any gait-altering pathology |
| | Cognitive impairment or inadequate English language skills |
| | that interfere with patient's ability to give fully informed |
| | consent or complete the baseline or follow-up assessments |
| Lumbar | Inclusion criteria |
| spine | Clinical diagnosis of either lumbar spinal stenosis, |
| | mechanical low back pain, or lumbar disc herniation |
| | Aged greater than 18 |
| | Exclusion criteria |
| | Women who are pregnant |
| | Serious spinal pathology including cancer, infection, cauda |
| | equina syndrome, spinal fracture, inflammatory arthritis |
| | Present with active Paget's disease of the spine |
| | Previous lumbar spinal surgery |
| | Presence of significant lumbar scoliosis (Cobb |
| | angle >25°) or other spinal deformities |
| | Meyerding classification grade 2 or greater spondylolisthesis |
| | Symptomatic hip disease with symptoms reproduced with |
| | external or internal rotation of the hip joint |
| | Inability to walk independently without the use of a walking |
| | aid or investigator assistance |
| | Cognitive impairment or inadequate English language skills |
| | that interfere with patient's ability to give fully informed |
| | consent or complete the baseline or follow-up assessments |
| Faller | Inclusion criteria |
| | Primary diagnosis of “falls in the last week for investigation” |
| | Assessed by an experienced neurosurgeon and geriatrician |
| | to have a clearly severe walking impairment |
| | Aged greater than 18 |
| | Exclusion criteria |
| | Inability to walk independently without the use of a walking |
| | aid or investigator assistance |
| | Cognitive impairment or inadequate English language skills |
| | that interfere with patient's ability to give fully informed |
| | consent or complete the baseline or follow-up assessments |

### 2.2 Procedure

All subjects completed a pre-assessment questionnaire to obtain demographic data. In the spine cohort, baseline levels of disability (Oswestry Disability Index and Visual Analogue Scale) were also obtained as part of a standardised clinic assessment. All subjects underwent an unobstructed walk along a straight pathway at a comfortable pace for a self-selected distance. Prior to the walk, participants were fitted at the sternal angle (FIG. 1) with the inertial measurement unit: MetaMotion@ (MMC) manufactured by Mbientlab Inc. (California, USA). Following a short initial pause to orient the MMC device, participants walked a self-selected distance (15-120 m) along an unobstructed pathway on level ground. Trials were discarded if the patient did (or could) not pause to orient the device, walk more than 15 m or required a walking aid during the bout.

In this example the MMC device recorded the entire walking bout, and the data captured was transmitted via Bluetooth™ to an Android™ smartphone running the IMUGait Recorder application developed for this study. The IMUGait Recorder application then uploaded the raw data to a centralised database where a customised python script was used to process the gait metrics for that walking bout. The IMUGaitPy program was then used for gait detection and extraction of gait features to calculate relevant gait metrics. Although the MMC can capture a large collection of metrics, only gait velocity, cadence, and stride length were considered for this study.

### 2.3 Statistical Analysis

Demographic data and gait metrics were evaluated for normality using a Shapiro-Wilk Test. Two-tailed independent t-tests were performed in the setting of normally distributed variables. The Chi-square Test of Independence was used to investigate relationships between categorical demographic variables. In the settings of non-normal variables data has been reported using the standard median and inter-quartile range (IQR). For the purposes of comparing distributions of non-normal data the Mann-Whitney U test was implemented. A Pearson's correlation chi-square test was performed to compare correlation between the iMoS™ and standardised PROM data. Statistical significance was considered with a p-value<0.05. All statistics were completed using IBM SPSS v26.0 (IBM, Armonk, NY).

### 2.4 Demographic Information of Participants

176 subjects were included in the study including 75 health controls, 85 lumbar spine pathology subjects, and 16 fallers admitted with a primary diagnosis of “falls in the last week for investigation” (all with a severe walking impairment as assessed by an experienced geriatrician and neurosurgeon, where 7 out of 16 patients had at least 5 falls in the previous week). Baseline demographics are shown in Table 7. There were baseline differences in continuous demographic variables between all study population groups. Between the healthy and spine cohorts, mean age (43.4 vs 56.1 respectively; p<0.001) and body mass index (BMI) (25.2 vs 27.8; p<0.001) were significantly different. Between the healthy and faller cohorts, mean age was significantly different (43.4 years vs 70.1 years respectively; p<0.001) while BMI was similar (25.2 vs 25.7 respectively; p=0.715). Between the spine and faller cohorts, mean age (56.1 vs 70.1 respectively; p=0.002) was significantly different while BMI (27.8 vs 25.7 respectively; p=0.096) was similar. Categorical variables (gender distribution, diabetic status, smoker status) were similar across all groups.

TABLE 7
Demographic features of the healthy controls
and spine patients included in the analysis.

| | | |
|---|---|---|
| Healthy controls | Lumbar spine | Faller |
| (n = 75) | (n = 85) | (n = 16) |

Continuous (Mean [Range, (SD)])

| | | | |
|---|---|---|---|
| Age (years) | 43.4 | 56.1 | 70.1 |
| | [21-91, (16.4)] | [23-86, (15.4)] | [43-89, (17.1)] |
| BMI (kg/m² | 25.2 | 27.8 | 25.7 |
| | [17.7-39.9, | [19.5-37.5, | [19.0-35.4, |
| | (4.02)] | (4.36)] | (5.57)] |

Categorical (n =, [% of total])
Gender

| | | | |
|---|---|---|---|
| Male | 33 [44] | 47 [55] | 8 [50] |
| Female | 42 [56] | 38 [45] | 8 [50] |
| Diabetic | 2 [3] | 4 [5] | 0 [0] |
| Smoker | 6 [8] | 8 [9] | 1 [7] |

BMI = body mass index; n = number of data entries for the respective category.

2.5 Gait Metrics are Significantly Different Between Patients with a Gait-Altering Pathology and Healthy Controls

Mean values of gait metrics (gait velocity, cadence, and stride length) were statistically significantly lower in spine (1.13 m/s, 106 steps per minute (median), and 1.27 m, respectively; p<0.001) and falls (0.637 m/s, 84.5 steps per minute, and 0.898 m, respectively; p<0.001) cohorts compared to healthy controls (1.38 m/s, 116 steps per minute (median), and 1.42 m, respectively). When these cohorts were compared between age brackets (21-40 years, 41-60 years, and 61+ years), these differences retained statistical significance except between the mean stride length of healthy controls and the spine cohort in all age brackets (p=0.102-0.336), and between lumbar spine patients and healthy controls in the 61+ years age bracket (p=0.083-0.336).

### 2.6 Cadence and Stride Length are the Two Best Simple Gait Metrics to Determine Walking Impairment

After scaling each metric to a value out of 10 (upper and lower cut-offs for cadence and stride length as in Table 1; lower cut-off for gait velocity=0.2 m/s, upper cut-off for gait velocity=1.4 m/s, so that the magnitude of a metric would not bias its contribution, binary logistic regression analysis revealed that cadence and stride length were the pair of metrics with the highest accuracy (77.8%) in correctly differentiating healthy walking (healthy controls) from pathological walking (lumbar spine and faller cohort) (in Table 8). Coefficients of −1.21 (cadence) and −0.453 (stride length) indicate that cadence had more influence in this model, by a factor of approximately 2.7. If given weightings according to this and combined to form a score out of 100 points, cadence should be allocated 73 points and stride length 27 points. A score with this composition results in an area under the receiver operating characteristic curve (AUC) of 0.850 indicating good stratification. To enhance simplicity and convenience, cadence was allocated 70 points and stride length 30 points, without compromising on stratification ability (still, AUC=0.850). This scoring arrangement is how the iMoS is determined.

TABLE 8
Different regression models trialled for the differentiation
between normal and abnormal gait.

| Combination of | Nagelkerke | Accuracy | | |
|---|---|---|---|---|
| gait metrics | R Square | (%) | Coefficient | P-value |
| Gait velocity* | 0.483 | 77.8 | −0.037 | 0.951 |
| Cadence | | | −1.19 | 0.003 |
| Stride length* | | | −0.43 | 0.36 |
| Gait velocity | 0.479 | 76.7 | −0.563 | <0.001 |
| Cadence | | | −0.893 | <0.001 |
| Gait velocity^{†} | 0.432 | 75.6 | −1.63 | <0.001 |
| Stride length^{†} | | | 0.799 | 0.002 |
| Cadence | 0.483 | 77.8 | −1.21 | <0.001 |
| Stride length | | | −0.453 | <0.001 |
| Gait velocity | 0.371 | 72.7 | −0.775 | <0.001 |
| Cadence | 0.402 | 75.6 | −1.13 | <0.001 |
| Stride length | 0.213 | 64.2 | −0.449 | <0.001 |

*, ^{†}These metrics have significant multicollinearity, weakening the statistical power of the regression models produced.

### 2.7 Correlation Between the Immediate Mobility Score and Patient-Reported Outcome Measures

Pearson correlation analysis revealed a moderate correlation between the iMoS and ODI (r=−0.570; p<0.001), and a weak correlation between the iMoS and VAS (r=−0.351; p=0.01). These relationships are summarised in Table 9.

TABLE 9
Correlations between objective and subjective
metrics for lumbar spine patients.

| Metrics | ODI | VAS | iMoS ™ |
|---|---|---|---|
| ODI | 1.00 | | |
| VAS | 0.545 | 1.00 | |
| iMoS ™ | −0.493 | −0.334 | 1.00 |

All correlations are significant (p < 0.05). ODI = Oswestry Disability Index; VAS = Visual Analogue Scale; iMoS = Immediate Mobility Score.

### 2.8 the Immediate Mobility Score can Distinguish Between Differing Degrees of Walking Impairment

The three cohorts in this study (healthy, lumbar spine, faller) form three different degrees of walking performance. The healthy cohort represents normal walking performance for different age brackets. The spinal cohort represents some degree of walking impairment, though not as severe as the faller cohort, wherein 7 out of 16 patients had at least 5 falls within the previous week. When comparing iMoS scores between healthy participants and spinal patients, significant differences were found for all age brackets with median and interquartile range values of 93.5 (87.9-99.1) vs 71.8 (60.5-83.2) for the 21-40 years age group (p<0.001), 95.2 (91.2-99.7) vs 81.0 (68.3-93.7) for the 41-60 years age group (p<0.001), and 84.0 (78.6-89.4) vs 72.8 (60.9-84.7) for the 61+ years age group (p=0.03). Similarly, significant differences were found between iMoS scores of spinal patients and fallers. Median and interquartile range values for iMoS™ scores in the faller cohort were 40.7 (18.9-62.6) for the 41-60 years age group (p<0.001), and 39.5 (23.7-55.4) for the 61+ years age group (p<0.001). These values are summarized in Table 10.

TABLE 10
Comparison of iMoS scores between groups.

| | | | | Significance of | Significance of |
|---|---|---|---|---|---|
| | | | | difference with | difference with |
| | Age | Number of | iMoS ™ | healthy controls | spine patients |
| Group | bracket | participants | Median (IQR) | P-value* | P-value^{†} |
| Healthy | 21-40 | 45 | 93.5 (87.9-99.1) | | |
| controls | 41-60 | 23 | 95.2 (91.2-99.7) | | |
| | 61+ | 7 | 84.0 (78.6-89.4) | | |
| | All | 75 | 93.9 (88.6-99.2) | | |
| Lumbar | 21-40 | 11 | 71.8 (60.5-83.2) | <0.001 | |
| spine | 41-60 | 37 | 81.0 (68.3-93.7) | <0.001 | |
| | 61+ | 37 | 72.8 (60.9-84.7) | 0.0029 | |
| | All | 85 | 80.7 (71.2-90.2) | <0.001 | |
| Faller | 21-40 | 0 | | | |
| | 41-60 | 5 | 40.7 (18.9-62.6) | <0.001 | 0.001 |
| | 61+ | 11 | 39.5 (23.7-55.4) | <0.001 | <0.001 |
| | All | 16 | 40.5 (24.2-56.6) | <0.001 | <0.001 |

iMoS = Immediate Mobility Score; IQR = interquartile range.
*P-value represents significance of difference between patient cohort and healthy controls for each age bracket.
^{†}P-value represents significance of difference between faller cohort and lumbar spine cohort for each age bracket.

Wearable devices have strong potential for widespread integration into health care services. However, a barrier described by previous studies is regarding difficulties in maintaining long-term compliance. For instance, a study using wearable sensors to remotely monitor vital signs over an average period spanning 84 days reported the loss of 10-30% of data points across most patients. This issue is not applicable to the iMOS as a rapid assessment at a single time-point, where lengthy assessment times and long-term compliance are not needed, enhancing its practicality.

Some doubt exists regarding the accuracy and reliability of IMUs when measuring more nuanced metrics such as asymmetry and variability, with one study where the output for gait symmetry was omitted altogether due to the heel strike detection algorithm being inadequate, and another where ICC scores for step time variability and step length variability were below 0.4, indicating poor reliability. This demonstrates another benefit of the iMoS, which only relies on simple gait metrics that have been shown to be accurately and reliably measured by IMUs in the literature. Overall, wearable devices have significant potential for largescale incorporation into health care services.

Owing to its simplicity, the iMoS can be calculated using both wearable devices and manual methods. While gait analysis in the present study was performed using a wearable sensor, the metrics gait velocity and cadence can also be obtained manually, with the clinician observing a walking bout and using a stopwatch and pedometer. An additional consideration is the minimum amount of distance that is required to reliably measure stride length and cadence. Recent studies have concluded that as little as three gait cycles (three steps with each foot) could be used to reliably measure simple spatiotemporal gait metrics (such as stride length and cadence) using a wearable sensor (with intraclass correlation coefficient values exceeding 0.9). This indicates that iMoS assessments using a wearable sensor can be performed over distances as short as five metres, signifying utility for patients with severe gait-altering pathologies who may not be able to walk farther. Yet, this study was performed using wearable sensors—manually calculated iMoS scores are likely to be inaccurate at shorter distances. Additionally, diseases where gait deteriorates over the duration of a walking bout, such as lumbar spinal stenosis, may not present with a clinically observable deficit at short distances. Therefore, the optimal walking distance for iMoS assessment varies according to the clinical scenario. Wearable devices facilitate the remote monitoring of gait and walking metrics and a real-time iMoS score can be computed and streamed continuously to health care providers, providing alerts if acute deterioration is detected.

### Example 3: Gait Symmetry Index (GSi)

The GSi algorithm provides an objective tool to assess walking symmetry with a scoring range of 0 (highly asymmetric) to 100 (‘normal’ gait symmetry). The GSi reflects deviation from mean normative values for each gait metric. The normative values are acquired from wearable sensor-based objective data capture in a control population. Ggait velocity, step length asymmetry and step time asymmetry are relevant metrics to be considered when assessing walking asymmetry (Table 11). Due to the significant correlation of gait velocity with functional disability in various gait-altering pathologies, a slightly higher weighting was allotted in the scoring algorithm (Table 12).

TABLE 11
Relevant metrics in GSi

| | Normative Values | Scoring | |
|---|---|---|---|
| Metric | (Mean ± SD) | Range | Score |
| 1. Gait velocity (m/s) | 1.43 ± 0.18 | 0-1.4 | 0-40 |
| 2. Step time | 31.6 ± 16.2 | >32 | 0-30 |
| asymmetry (ms) | | | |
| 3. Step length | 53.7 ± 20.1 | >54 | 0-30 |
| asymmetry (cm) | | | |

| | |
|---|---|
| SGi total | 100 |

TABLE 12
Scoring of GSi

| Gait Velocity (GV) | | Step time asymmetry (STA) | | Step length asymmetry (SLA) | |
|---|---|---|---|---|---|
| GV < 1.4 m/s | $\{ \frac{GV}{1.4} \} \times 40$ | STA > 32 ms | $\{ \frac{32}{STA} \} \times 30$ | SLA > 54 cm | $\{ \frac{54}{SLA} \} \times 30$ |
| GV > 1.4 m/s | 40 | STA < 32 ms | 30 | SLA < 54 cm | 30 |

The GSi was devised to objectify clinical gait assessment in unilateral gait disorders (e.g. stroke, sciatica, osteoarthritis). In particular, the GSi aims to evaluate walking asymmetry in the community or at-home (termed ‘free-living’ gait) with data extraction from a wearable device providing continuous, non-biased, and objective data stream of patient performance. Clinical performance of the proposed GSi was assessed in a prospective, non-randomised single surgeon series of 33 patients with LDH patients, by similar objective data capture using wearable inertial sensors.

GSi can be calculated using the following:

$$GV = \frac{Total distance}{Total time} = \frac{average SL}{average ST} = \frac{{\sum }_{i = 1}^{n} GV of {step}_{i}}{2 n} \qquad Equation 1$$

$$ST = \frac{{\sum }_{i = 1}^{n} {steptime}_{i}}{2 n} \qquad Equation 2$$

$$SL = \frac{{\sum }_{i = 1}^{n} = {stepleng}_{i}}{2 n} \qquad Equation 3$$

$$STA = \frac{{\sum }_{i = 2}^{n} ( {steptime}_{i} - ste {time}_{i - 1} )}{2 n} \qquad Equation 4$$

$$SLA = \frac{{\sum }_{i = 2}^{n} ( {steplength}_{i} - step {length}_{i - 1} )}{2 n} \qquad Equation 5$$

These calculations assume that n steps were taken over a given bout, with ‘i’ representing a specific step number (n). ST=step time, SL=stride length, GV=gait velocity, STA=step time asymmetry, SLA=step length asymmetry.

### 3.1 Study Participants

The participants of this study were patients presenting with radiating buttock and/or leg pain (sciatica). During their clinic visit, study parameters and risks were discussed, and consent obtained. Patients presenting with symptoms of radiating buttock and/or leg pain or ‘sciatica’, secondary to LDH were considered for inclusion. Exclusion criteria included infection, cancer, prior lumbar spine surgery at the index level, and presence of other potentially gait-altering pathologies including knee, hip or neurological dysfunction. Participants completed a participant questionnaire to obtain demographic and clinical information. Age-matched healthy participants were recruited from the community as controls in a 1:1 ratio for this study following a similar semi-structured interview.

### 3.2 Procedure

Prior to the walk, participants were fitted at the sternal angle (FIG. 1) with the inertial measurement unit: MetaMotion@ (MMC) manufactured by Mbientlab Inc. (California, USA). Following a short initial pause to orient the MMC device, participants walked a self-selected distance (15-120 m) along an unobstructed pathway on level ground. Trials were discarded if the patient did (or could) not pause to orient the device, walk a minimum of 15 m or required a walking aid during the bout.

### 3.3 Wearable Device

The MMC is a wearable sensor which contains a 16 bit 100 Hz triaxial accelerometer for the detection of linear acceleration (anteroposterior, mediolateral, and vertical), a 16 bit 100 Hz triaxial gyroscope for the detection of angular acceleration (pitch, roll and yaw), and a 0.3 μT 25 Hz triaxial magnetometer to assess orientation relative to the Earth's magnetic field (North-South). Following signal processing with a Kalman filter, captured data is stored as a matrix of the values corresponding to each time point (100 captures per second) for up to 20 minutes of walking.

### 3.4 Data Processing

For the purposes of this study, the MMC device recorded the entire walking bout, and the data captured was transmitted via Bluetooth™ to an Android™ smartphone running the IMUGait Recorder application developed specifically for this study. The IMUGait Recorder application then uploaded the raw data to a centralised database where a modified version of Czech et al's open-source python program (IMUGaitPy program) was used to process the gait metrics for that walking bout. The IMUGaitPy program was then used for gait detection and extraction of gait features across three domains (spatiotemporal, asymmetry and variability) to calculate relevant gait metrics including, gait velocity, step time, step length, stride length, step time asymmetry and step length asymmetry.

### 3.5 Sensor Accuracy

Accuracy performance of the MCC, inertial wearable sensor for spatial and temporal measurements (step count, gait velocity, step time, stride length, and step length) was assessed by comparison with videography-derived measurements. Bland-Altman plots were used to evaluate inter-method agreement for gait metrics captured during walking bouts of 30 metres and over in healthy controls (n=16).

### 3.6 Statistical Analysis

Data analyses were performed using Prism 9 (GraphPad Software). Normality was assessed using Shapiro-Wilk tests and inspection of histograms where necessary and statistical significance was considered for p-value<0.05. Descriptive statistics were calculated for demographic variables including; age, BMI, gender, presence of diabetes and smoking. Spatiotemporal parameters of gait were calculated, and step measurements chosen for calculations of gait asymmetry due to greater reliability being reported in literature, compared stride measurements (28). Differences in the aforementioned gait metrics and GSi scores between Lumbar Disc Herniation (LDH) participants (surgical management, conservative management and pooled groups) and control participants were identified using Kruskal-Wallis tests or one-way analysis of variance (ANOVA) tests. Post-hoc independent samples (two-tailed) t-tests and Mann-Whitney U tests were used to establish where these differences exist between these groups. P values<0.05 were considered statistically significant. Correlation of GSi scores with ODI and VAS Pain scores was assessed by simple linear regression.

### 3.7 Participant Demographics

A total of 66 participants met the inclusion for this observational study of gait over the study period comprising of 24 females and 42 males. 33 LDH participants were sub grouped into 14 surgical management and 19 conservative management with 33 age-matched controls recruited. Included participants were of mostly similar demographic characteristics (with the exception of gender) as seen in Table 13. The average age (mean+age) for the study cohort being 44+13 years (surgical: 44+9, conservative: 45+16).

TABLE 13
Demographic and clinical characteristics of participants.

| | | LDH | LDH Subgroups | | |
|---|---|---|---|---|---|
| | Healthy | (pooled) | Surgical | Conservative | P |
| Demographic | | | | | |
| N | 33 | 33 | 14 | 19 | |
| Age | 44 ± 13 | 44 ± 13 | 44 ± 9 | 45 ± 16 | 0.9965 |
| Female (%) | 17 (52) | 7 (21) | 2 (14) | 5 (26) | 0.0191 |
| BMI | 25 (18-37) | 27 (22-38) | 26 (23-38) | 27 (21-38) | 0.2909 |
| Smoking (%) | 1 (3) | 3 (9) | 2 (14) | 1 (5) | 0.5656 |
| Diabetes (%) | 2 (6) | 1 (3) | 0 (0) | 1 (5) | 0.7816 |
| Clinical | | | | | |
| Daily Step | N/A | 3500 | 2000 | 3700 | 0.8547 |
| Count | | (100-12000) | (100-12000) | (1000-10000) | |
| Oswestry | N/A | 42.2 + 21.6 | 47.7 + 22.7 | 37.8 + 20.2 | 0.4677 |
| Disability | | | | | |
| Index | | | | | |
| VAS Pain | N/A | 6.1 + 2.4 | 6.6 + 2.5 | 5.7 + 2.4 | 0.3729 |
| Score | | | | | |
| Diagnosis | | | | | 0.6681 |
| (Level) | | | | | |
| Multi | N/A | 2 | 2 | 0 | |
| (L5/S1, L4/5) | | | | | |
| L5/S1 | N/A | 11 | 4 | 7 | |
| L4/5 | N/A | 8 | 3 | 5 | |
| L3/4 | N/A | 2 | 0 | 2 | |
| L2/3 | N/A | 2 | 0 | 2 | |

Normally distributed data analysed using one-way ANOVA is displayed as (mean + standard deviation) while non-parametric analysis using Kruskal Wallis test is displayed as (median (minimum-maximum)). Statistical significance set at p ≤ 0.05. P value in the table represents differences in mean/medians (according to analysis of variance) or differences in proportions (according to chi-square tests).
BMI = Body Mass Index. VAS = Visual Analogue Scale.

The average daily step count of LDH participants was 3500 (range, 100-12000) with ODI of 42.2+21.6 (mean+SD) and VAS pain score of 6.1+2.4. Single-level disc herniation diagnoses comprised a range of index levels including L5/S1 (11), L4/5 (8), L3/4 (2) and L2/3 (2). 2 LDH participants had multi-level disc herniations (L4/5 and L5/S1). Although these preoperative characteristics were on average worse in the operative management subgroup compared to the conservative management subgroup, these differences were not statistically significant.

### 3.8 Gait Metrics

Spatiotemporal parameters including gait velocity (p<0.0001), step length (p<0.0135) and step time (p<0.0001) along with asymmetry parameters for step time (p=0.0227) and step length (p=0.0071) were significantly different between LDH and controls (Table 14). LDH participants have a typical gait pattern of lower gait velocity (−20.3%) lower step length (−9.47%) whilst step time (+10.6%), step time asymmetry (+23.1%) and step length asymmetry (+39.1%) are increased. These deteriorations in gait parameters were greater in the surgical management subgroup, compared to the conservative management subgroup. Mean bias during accuracy assessment of the inertial wearable sensor ranged from 2.84%-0.25% (Table 15).

TABLE 14
Accuracy performance of MMC, wearable accelerometer in comparison
to videography-derived spatial and temporal gait metrics.
Bland-Altman plots were used to evaluate inter-method agreement
for gait metrics captured during walking bouts of 30 metres
and over in healthy controls (n = 16).

| | Bias (% Difference): | | 95% Confidence Interval | |
|---|---|---|---|---|
| | Sensor-Videography | | Upper | Lower |
| | Mean | SD | Bound | Bound |
| Steps | 0.775 | 2.82 | 6.31 | −4.76 |
| Gait Velocity | 2.84 | 10.5 | 23.5 | −17.8 |
| Step Length | 2.26 | 10.5 | 22.9 | −18.4 |
| Step Time | −0.250 | 2.17 | 4.00 | −4.50 |

TABLE 15
Gait metrics of participants derived from wearable device.

| | LDH | | | | |
|---|---|---|---|---|---|
| Healthy | Pooled | Surgical | Conservative | | |
| (n = 33) | (n = 33) | (n = 14) | (n = 19) | F/H | P |

Spatial and Temporal Metrics

| | | | | | | |
|---|---|---|---|---|---|---|
| Gait | 1.43 ± | 1.14 ± | 1.06 ± | 1.21 ± | 12.4 | <0.0001 |
| Velocity (m/s) | 0.182^{P, S, C} | 0.260^{H} | 0.328^{H} | 0.180^{H} | | |
| (control − | | −20.3% | −25.9% | −15.4% | | |
| difference) | | | | | | |
| Step Length | 72.9 ± | 66.0 ± | 61.9 ± | 69.0 ± | 3.75 | 0.0135 |
| (cm) | 10.2^{P, S} | 12.0^{H} | 13.1^{H} | 10.6 | | |
| (control − | | −9.47% | −15.1% | −5.35% | | |
| difference) | | | | | | |
| Step Time | 0.519 (0.415- | 0.574 (0.497- | 0.581 (0.501- | 0.574 (0.497- | 30.2 | <0.0001 |
| (s) | 0.590)^{P, S, C} | 0.889)^{H} | 0.889)^{H} | 0.661)^{H} | | |
| (control − | | +10.6% | +12.0% | +10.6% | | |
| difference) | | | | | | |

Asymmetry

| | | | | | | |
|---|---|---|---|---|---|---|
| Step Time | 29.0 (11.9- | 35.7 (12.6- | 73.0 (22.1- | 34.2 (12.6- | 9.56 | 0.0227 |
| Asymmetry (ms) | 70.2)^{P, S} | 425)^{H} | 425)^{H} | 155) | | |
| (control − | | +23.1% | +51.7% | +17.9% | | |
| difference) | | | | | | |
| Step Length | 4.86 (2.54- | 6.76 (3.25- | 9.94 (3.44- | 5.37 (3.25- | 12.1 | 0.0071 |
| Asymmetry (cm) | 9.71)^{P, S} | 33.6)^{H} | 15.0)^{H, C} | 33.6)^{S} | | |
| (control − | | +39.1% | +105% | +10.5% | | |
| difference) | | | | | | |

Gait Symmetry Index (GSi)

| | | | | | | |
|---|---|---|---|---|---|---|
| GSI | 99.8 (70.5- | 83.1 (28.9 - | 61.2 (28.9- | 89.2 (36.9- | 21.3 | <0.0001 |
| (score/100) | 100.0)^{P, S, C} | 100.0)^{H} | 100.0)^{H} | 100.0)^{H} | | |
| (control − | | −16.7% | −38.7% | −10.6% | | |
| difference) | | | | | | |

Normally distributed data analysed using one-way ANOVA is displayed as (mean + standard deviation) while non-parametric analysis using Kruskal Wallis test is displayed as (median (minimum-maximum)). P value in the table represents difference between groups derived from Kruskal Wallis tests or ANOVA. Annotations within table represent differences between groups and are interpreted as follows: H = different from healthy controls, P = different from pooled LDH, S = different from surgical LDH, C = different from conservative LDH. Statistical significance set at p ≤ 0.05 (bolded). m = metre, s = second. ms = millisecond.

### 3.9 GSi Scores

Walking asymmetry according to GSi was significantly different across control and LDH participants (p<0.0001). GSi scores (median, range) were lower in LDH participants (83.1, 28.9-100.0) compared to controls (99.8, 70.5-100). These differences in GSi scores between the surgical management (61.2 (28.9-100.0) and conservative management (89.2 (36.9-100.0) subgroups provided a large range to identify, assess and monitor the walking asymmetry of LDH participants. Moreover, GSi scores also correlated with patient-reported outcome measures (Table 16) such as the ODI, with a slope of −0.7345 (r squared=0.5325, p<<0.0001). This correlation was also present with VAS Pain Scores (albeit weaker), with a slope of −4.021 (r squared=0.2049, p=0.0082).

TABLE 16
Correlations for GSi and patient-reported outcome measures.

| | Slope | 95% CI | R² | P value |
|---|---|---|---|---|
| ODI (n = 29) | −0.7345 | −1.006 to −0.4627 | 0.5325 | <0.0001 |
| VAS Pain Score | −4.021 | −6.923 to −1.119 | 0.2049 | 0.0082 |
| (n = 33) | | | | |

GSi = Gait Symmetry index;
ODI = Oswestry Disability Index,
VAS = Visual Analogue Scale

### 3.10 Discussion

The GSi provides objective gait data retrieved from prolonged wearable based assessment tracking multiple gait cycles and significant distance (˜100 m) and is a holistic assessment of functional ability compared to patient questionnaires which provides a “snapshot” of health status and are subjective by their very nature.

In this example the examination of several gait metrics has been effective in detecting the expected significant gait differences between the LDH and control population. This translates in the LDH population, to a lower gait velocity (median: −22.6%), step length (median: −12.3%) and cadence (median: −12.2%) and a corresponding increased step time (+10.8%).

Most notably, however LDH participants experience greater gait asymmetry both in terms of step time (+70.0%) and step length (+51.6%), warranting interest in the development of the GSi. This is consistent with the observation that as patients experience worse symptoms unilaterally, they may try to over-correct gait on the corresponding side to limit time spent loading the symptomatic side and exacerbating pain. It is expected that similar findings would be expected with other unilateral pathologies including arthritic joints, cerebrovascular accident, or myopathy.

The GSi is an index with easy interpretation, specifically designed for the clinical setting as a clinical decision-making adjunct. Although not specific for the LDH setting it represents a sensitive measure to detect individuals that may require further investigation or intervention to restore a closer to ‘normal’ (and symmetric) gait pattern. Although more finite and precise scoring systems may be calculated, a simple algorithm was opted to enable convenient clinical use. This is of key importance as it allows the index to be rapidly reproduced (even manually) and communicated between relevant members of the clinical setting (e.g. health member and patient).

The GSi's key strength lies in that it is sensitive at detecting LDH-associated abnormalities in gait symmetry not only amongst surgical patients experiencing debilitating symptoms but also non-operative patients with more tolerable symptoms. Given this GSi distribution in the pathological LDH population, a score of less than “88” (lower threshold of inter-quartile range in normative population) is a clinically pertinent cut-off. Subjects with GSi<88, warrant consideration for some form of intervention (rather than conservative management), though the nature and degree ultimately depend on the underlying cause of gait change. At present this work demonstrates efficacy at detecting gait abnormalities however ongoing research is required to assess its diagnostic utility and other clinical uses, especially in other unilateral gait-altering pathologies. As a repeated measure there is potential for its use in the setting of reassessing gait deficits during rehabilitation and post-surgical follow-up.

### Example 4: Combined Mobility Score (CMoS)

### 4.1 Study Participants

A cohort of patients with spine, hip and knee, and neurological pathologies, were recruited. An additional cohort of falls patients who were judged by an experienced geriatrician and neurosurgeon to have severe walking impairment were recruited. Healthy control subjects were recruited from the community using verbal outreach. For each participant, study parameters and risks were discussed, and consent obtained.

### 4.2 Procedure

All subjects completed a pre-assessment questionnaire to obtain demographic data. In the spine, hip and knee cohort, baseline levels of disability (Oswestry Disability Index and Visual Analogue Scale) were also obtained as part of a standardised clinic assessment. All subjects underwent an unobstructed walk along a straight pathway at a comfortable pace for a self-selected distance. Prior to the walk, participants were fitted at the sternal angle with the inertial measurement unit: MetaMotion@ (MMC) manufactured by Mbientlab Inc. (California, USA). Following a short initial pause to orient the MMC device, participants walked a self-selected distance (15-220 m) along an unobstructed pathway on level ground. Trials were discarded if the patient did (or could) not pause to orient the device, walk more than 15 m or required a walking aid during the bout.

### 4.3 Wearable Device

The MMC is a wearable sensor which contains a 16 bit 100 Hz triaxial accelerometer for the detection of linear acceleration (anteroposterior, mediolateral, and vertical), a 16 bit 100 Hz triaxial gyroscope for the detection of angular acceleration (pitch, roll and yaw), and a 0.3 μT 25 Hz triaxial magnetometer to assess orientation relative to the Earth's magnetic field (North-South). Following signal processing using a Kalman filter, the captured data is stored as a matrix of the values corresponding to each time point (100 captures per second) for up to 20 minutes of walking.

For the purposes of this study, the MMC device recorded the entire walking bout, and the data captured was transmitted via Bluetooth™ to an Android™ smartphone running the IMUGait Recorder application developed for this study (FIG. 2). The IMUGait Recorder application then uploaded the raw data to a centralised database where a customised python script was used to process the gait metrics for that walking bout. The IMUGaitPy program was then used for gait detection and extraction of gait features to calculate relevant gait metrics and the WORM algorithm.

### 4.4 Statistical Analysis

Demographic data and gait metrics were evaluated for normality using a Shapiro-Wilk Test. Two-tailed independent t-tests were performed in the setting of normally distributed variables. The Chi-square Test of Independence was used to investigate relationships between categorical demographic variables. In the settings of non-normal variables data has been reported using the standard median and interquartile range (IQR). For the purposes of comparing distributions of non-normal data the Mann-Whitney U test was implemented. A Pearson's correlation chi-square test was performed to compare correlation between the CMOS and standardised PROM data. Statistical significance was considered with a p-value<0.05. All statistics were completed using IBM SPSS v26.0 (IBM, Armonk, NY).

### 4.5 Results

201 subjects were included in the study including 74 healthy controls, 122 lumbar spine pathology subjects, 20 hip pathology subjects, 11 knee pathology subjects and 16 (recent) fallers.

Gait metrics were significantly different between patients with a gait-altering pathology and healthy controls

Mean values of gait metrics were statistically significantly lower in hip, knee and spine (1.13 m/s, 106 steps per minute (median), and 1.27 m, respectively; p<0.001) and falls (0.637 m/s, 84.5 steps per minute, and 0.898 m, respectively; p<0.001) cohorts compared to healthy controls (1.38 m/s, 116 steps per minute (median), and 1.42 m, respectively). When these cohorts were compared between age brackets (21-40 years, 41-60 years, and 61+ years), these differences retained statistical significance except between the mean stride length of healthy controls and the spine cohort in all age brackets (p=0.102-0.336), and between lumbar spine patients and healthy controls in the 61+ years age bracket (p=0.083-0.336).

### 4.6 the Combined Mobility Score can Distinguish Between Healthy and Impaired Walking.

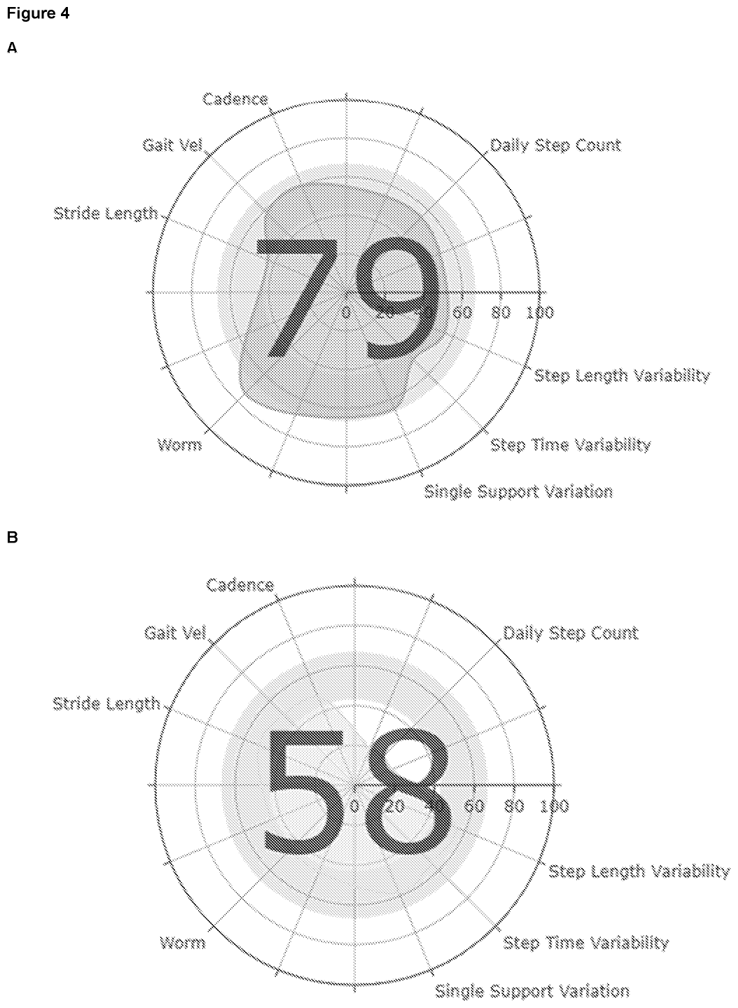

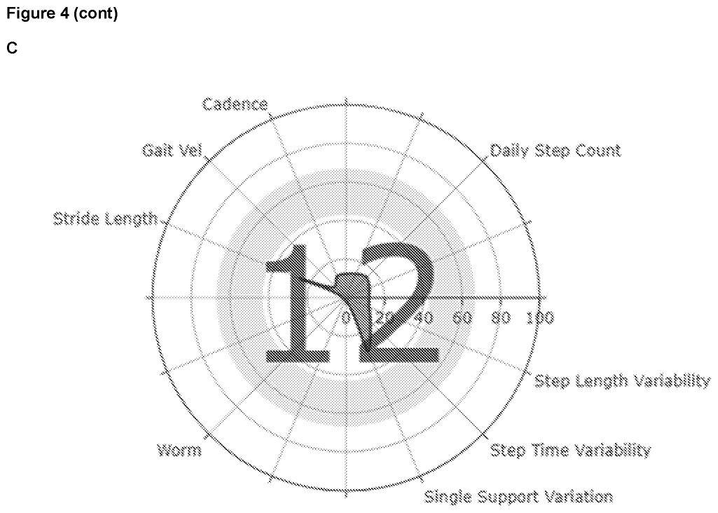

The CMOS was able to accurately distinguish between healthy controls and pathological cohorts, with an area under the receiver operating characteristic curve of 0.850 (indicating good stratification) as seen in FIG. 4.

### 4.7 the Combined Mobility Score can Act as a Scoping Tool to Distinguish Between Differing Causes of Walking Impairment.

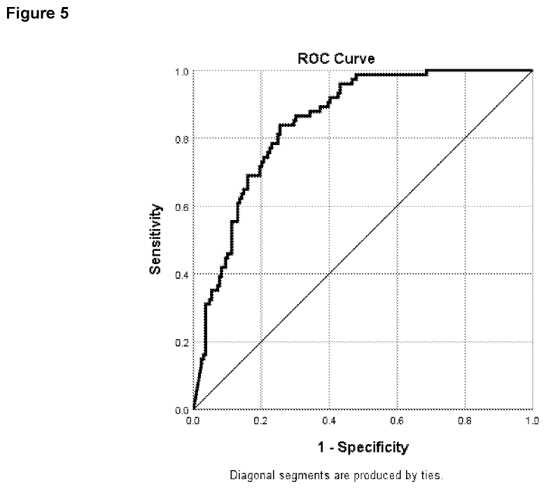

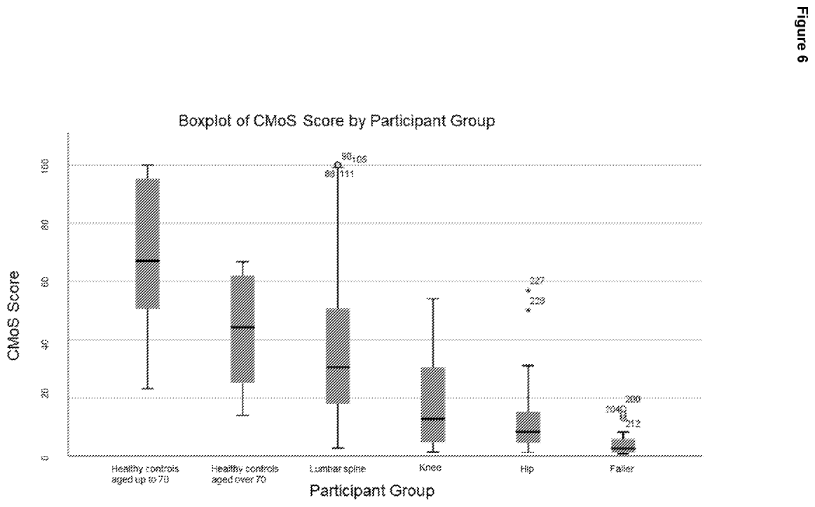

The distribution (mean+/−standard deviation) of CMOS for each patient group was significantly varied across patient groups. While the normal group averaged typically higher (68+/−25), patient groups such as lumbar spine (37+/−26), knee (20+/−19), hip (14+/−16) and fallers (5+/−5) averaged consistently lower. From this distribution we propose a classification system to stratify mobility health as seen below (Table 17, FIG. 5).

TABLE 17
Mobility health according to CMOS

| | Mobility health according to CMOS | | | | |
|---|---|---|---|---|---|
| | | | Mild | Moderate | Severe |
| Participant | Excellent | Normal | impairment | impairment | impairment |
| group | (81-100) | (61-80) | (41-60) | (21-40) | (0-20) |
| Healthy control | 27 | 20 | 17 | 9 | 1 |
| (under 70 yrs) | | | | | |
| (over 70 yrs) | | | | | |
| Lumbar spine | 12 | 12 | 23 | 32 | 43 |
| Faller | 0 | 0 | 0 | 0 | 16 |
| Hip | 0 | 0 | 2 | 3 | 15 |
| Knee | 0 | 0 | 2 | 3 | 6 |

### 4.8 Discussion

CMOS was developed to allow a comprehensive objective estimation of gait health, and to represent this in both numerical (a quantitative score out of 100) and visual (“gait cloud” or “gait signature”) formats. The CMOS takes into account the many aspects of gait including quality, quantity, consistency, and stability/balance with a single wearable accelerometer system that can be worn continuously.

Quantifying various aspects of human gait into a summative score provides an objective measure of the presence and extent of a pathological gait. One of the primary aims of the CMOS tool is its function as a scoping tool to identify gait deficits and categorise them based on disease-specific gait patterns. The pattern of walking impairment across each gait category-termed “gait signature”—can be visually appreciated to obtain insight about the patient's health and guide clinical decision-making. For instance, a patient with relatively normal gait quality and gait quantity but impaired gait consistency and gait stability may be suffering from a pathology which unilaterally affects mobility—the patient may benefit from a walking aid to support the affected side. A patient exhibiting severe gait deficiencies in all four categories (“severe global gait impairment”) may not be able to walk safely at all and may require a wheelchair. A gait signature showing a deficiency in gait quantity without compromise in any of the other categories indicates a sedentary lifestyle where overall gait health may be affected by a lack of motivation rather than any underlying pathology. Additional studies could investigate the gait signatures of other diseased populations, allowing for distinctions to be made between the gait signatures of patients with different pathologies. We envision this leading to the construction of “disease-specific gait signatures”, which is the characteristic pattern of gait alteration as visualised on a CMOS™ Wheel. This allows matching a patient's CMOS™ gait signature to a disease-specific gait signature to aid in the clinical diagnosis of gait-altering conditions. This also benefits patient rehabilitation and postoperative monitoring where clinicians could track a patient's gait signature over time as a deviation from (deterioration) or return to (improvement) the normative torus.

The CMOS Wheel (for example as represented in FIG. 3) complements this quantitative proxy measure of health status and visually defines exactly which aspect of gait has been altered by disease processes. Patients may benefit from an overall insight into a vital measure of their health: walking performance while clinicians can observe which specific aspect(s) of their patient's gait is impaired. This information may guide timely implementation of mobility interventions, falls prevention or fitness regiments that may be needed to specifically address these aspects of gait (for instance stability, fitness, symmetry, or quality).

One application of CMOS may be the interpretation of human gait as a clinical biomarker to track decline and recovery of health status. Although an individual's gait may decline with the onset of disease, it may also improve with recovery from disease, or following a specific treatment or with improvements in health and fitness from regular physical activity. The continuous tracking of CMOS scores yields various uses to the patient and clinician, by enabling the tracking of improvements in health status over time, leveraged by the continuous data stream provided by wearable sensors of gait performance. These include the screening of emerging disease processes, early detection of complications, and rehabilitating recovery to a relatively normative health state.

The recent pandemic has triggered a paradigm shift in the way society functions, as many jobs convert to productive remote work arrangements. Similar changes may arise within the health care setting with the increased uptake of telehealth practices and wearable sensors. In this study, a wearable sensor was used to demonstrate a comprehensive walking assessment method using the CMOS to distinguish between differing levels of walking impairment. Across all age brackets, CMOS scores were significantly lower in the spine, hip and knee cohorts and significantly lower in the faller cohort. Composed of multiple gait metrics—this score can be integrated into wearable devices containing accelerometers, to distinguish between these pathologies and potentially also gauge symptom severity.

Wearable devices have strong potential for widespread integration into health care services. However, a known barrier to their adoption is maintaining long-term compliance. This demonstrates another benefit of the CMOS, which requires as few as 6 gait cycles to calculate the CMOS score, although daily step count is still required to complete the score.

An additional consideration is the accuracy of wearable sensors for gait analysis.

One consideration is the amount of distance that is required to reliably measure stride length and cadence. It is known that as little as three gait cycles (three steps with each foot) could be used to reliably measure simple spatiotemporal gait metrics (such as stride length and cadence) using a wearable sensor (with intraclass correlation coefficient values exceeding 0.9). With the WORM score being able to be calculated from as few at 6 gait cycles. CMOS assessments using a wearable sensor can be performed over distances as short as five metres, signifying utility for patients with severe gait-altering pathologies who may not be able to walk farther. Additionally, diseases where gait deteriorates over the duration of a walking bout, such as lumbar spinal stenosis, patients may not present with a clinically observable deficit at short distances. Therefore, the optimal walking distance for CMOS assessment varies according to the clinical scenario and will be open to revision with future testing.

Another consideration is the weightings attributed to each component of mobility in the CMOS. As more data is collected understanding of how different disease-states result in specific deficits in walking performance and ability will inevitably improve. As this occurs, these weightings may be refined or more drastically revised to better optimize the sensitivity, specificity, and accuracy in the detection of disease-states.

Secondary algorithms may also be developed for other clinical purposes such as differentiating clinical severity or prediction of prognostic, post-intervention and/or recovery outcomes. As such, a clinical “decision-tree” tool may be developed (integrating artificial intelligence algorithms) to detect and classify patients accordingly: (1) initially into broader pathology groups (for instance spine, hip, knee) and (2) further into specific categories (for instance mild versus severe, surgical versus conservative, degenerative versus traumatic).

The CMOS is a clinical adjunct that effectively discriminates between normal and impaired walking, particularly in conditions affecting our biomechanical and/or neurological system, in addition to the general ageing process. A low CMOS score can alert clinicians that an intervention may be needed to prevent further injury from falls and improve mobility in the community. Although larger cohort studies are required, the CMOS has the potential to differentiate between differing levels of walking impairment in all real-life settings.

1. A system for determining a quantitative gait score, the system comprising:
a) an accelerometer configured to output signals indicative of movement of the subject along one or any combination of an x-axis, a y-axis, and a z-axis;
b) a magnetometer configured to output signals indicative of variations in position of the subject in a space defined by the x-axis, the y-axis, and the z-axis;
c) a gyroscope configured to output signals indicative of angular velocity of the subject around one or any combination of the x-axis, the y-axis, and the z-axis; and
d) a processor configured to receive the output signals and analyse the signals to determine the quantitative gait score for the subject from any combination of the gait metrics:
i) daily step count;
ii) step cadence;
iii) step time;
iv) step time asymmetry;
v) step length;
vi) stride length;
vii) step length asymmetry;
viii) single support time variability;
ix) walking orientation randomness metric (WORM Score); and
x) gait velocity or gait velocity variation;
wherein the x-axis is a horizontal axis to the ground directed forward of the subject's body; the y-axis being a horizontal axis to the ground directed laterally of the subject's body;
and the z-axis is vertical axis to ground.

2. The system of claim 1, wherein the accelerometer, magnetometer, gyroscope, and optionally the processor are in a sensor unit adapted to be disposed on the subject.

3. The system of claim 1, wherein the processor is configured to determine the quantitative gait score from any two or more gait metrics i-x.

4. The system of claim 3, wherein the quantitative gait score is at least one of:
i) SMoS calculated from gait velocity and daily step count;
ii) iMoS calculated from step cadence and stride length; and
iii) Gsi calculated from gait velocity or gait velocity variation, step length asymmetry and step time asymmetry; and
iv) Combined Mobility Score calculated from:
a) daily step count;
b) one or more of gait velocity, step length, stride length, and cadence;
c) one or more of step length asymmetry, step time asymmetry, single support time variability; and
d) walking orientation randomness metric (WORM Score).

5. A method for determining a quantitative gait score for a subject, the method comprising:
a) obtaining a gait velocity and daily step count for the subject, assigning a first numerical value to a walking speed, assigning a second numerical value to the daily step count and summing the first and second numerical values to provide the quantitative gait score;
wherein
if the walking speed is above a threshold walking speed, the first numerical value is a first maximum numerical value; or
if the walking speed is below the threshold walking speed, the walking speed is divided by the threshold walking speed to provide a product, and the product is multiplied by the maximum numerical value to provide the first numerical value;
and wherein
if the daily step count is above a threshold step count, the second numerical value is a second maximum numerical value; or
if the daily step count is below the threshold daily step count, the daily step count is divided by the threshold daily step count to provide a second product and the second product is multiplied by the second maximum numerical value to provide the second numerical value;
or
b) obtaining a step cadence and stride length for the subject and assigning a first numerical value to the step cadence, assigning a second numerical value to the stride length and summing the first and second numerical values to provide the quantitative gait score;
wherein
if the step cadence is below a first threshold cadence, the first numerical value is a first minimum numerical value; or
if the step cadence is above a second threshold cadence, the first numerical value is a first maximum numerical value; or
if the step cadence is between the first and second threshold cadences, the first threshold step cadence is subtracted from the step cadence and the difference between the step cadence and the first threshold step cadence is the first numerical value;
and wherein
if the stride length is below a first threshold stride length, the second numerical value is a second minimum numerical value; or
if the stride-length is above a second threshold stride length, the second numerical value is a second maximum numerical value; or
if the stride-length is between the first and second threshold stride-lengths, the second numerical value is calculated by multiplying the stride-length by a value equal to the value of the second threshold stride-length to provide a product and then subtracting half of the value from the product to provide the second numerical value; or
c) obtaining the gait velocity, step length asymmetry and step time asymmetry for the subject; assigning a score to each of the gait velocity, step length asymmetry and step time asymmetry, and summing the scores.

6. The method of claim 5, wherein the quantitative gait score is at least one of:
i) SMoS calculated from gait velocity and daily step count;
ii) iMoS calculated from step cadence and stride length;
iii) GSi calculated from gait velocity or gait velocity variation, step length asymmetry and step time asymmetry; and
iv) Combined Mobility Score calculated from:
e) daily step count;
f) one or more of gait velocity, step length, stride length, and cadence;
g) one or more of step length asymmetry, step time asymmetry, single support time variability; and
h) walking orientation randomness metric (WORM Score).

7. The method of claim 6, wherein the quantitative gait score is SMoS calculated from the sum of gait velocity and step length.

8. The method of claim 7, wherein if SmoS for the subject is 1.4 or less, the subject has a high fall risk.

9. The method of claim 7, wherein if SmoS for the subject is 1.5 or more, the subject has a low fall risk.

10. The method of claim 6, wherein the quantitative gait score is iMoS calculated from sum of cadence and stride length.

11. The method of claim 10, wherein if the iMoS for the subject is 95 or less, the subject has a high fall risk.

12. The method of claim 10, wherein if the iMoS for the subject is 100 or more, the subject has a low fall risk.

13. The method of claim 6, wherein the quantitative gait score is Gsi calculated from gait velocity or gait velocity variation, step length asymmetry and step time asymmetry.

14. The method of claim 13, wherein if the Gsi for the subject is 0.25 or more, the subject has a high fall risk.

15. The method of claim 13, wherein if the Gsi for the subject is 0.2 or less, the subject has a low fall risk.

16. The method of claim 6, wherein the quantitative gait score is the Combined Mobility Score calculated by:
a) obtaining gait metrics in gait categories i-iv:
i) daily step count;
ii) one or more of gait velocity, step length, stride length, and cadence;
iii) one or more of step length asymmetry, step time asymmetry, single support time variability; and
iv) walking orientation randomness metric (WORM Score);
b) assigning a numerical weighting to each gait category; and
c) summing the numerical weightings for the gait categories.

17. The method of claim 16, wherein if the WORM Score is above a threshold value, the numerical weighting assigned to it is not more than 5, 10, 15, or 20 percent of the total of the numerical weightings applied to all the gait categories.

18. The method of claim 16, wherein if the WORM Score is at or below a threshold value, the numerical weighting assigned to it is 20, 25, 20, 35, 40, 45 or 50 percent of the total of the numerical weightings applied to all the gait categories.
